(19) United States
(12) Patent Application Publication
(10) Pub. No.: US 2022/0047627 A1
(43) Pub. Date: Feb. 17, 2022

(54) NON-INFECTIVE NASAL SYMPTOM MANAGEMENT COMPOSITIONS AND METHODS

(71) Applicant: CMPD Licensing, LLC, Conroe, TX (US)

(72) Inventor: Jay Richard Ray, II, Conroe, TX (US)

(73) Assignee: CMPD Licensing, LLC, Conroe, TX (US)

(21) Appl. No.: 17/513,733

(22) Filed: Oct. 28, 2021

Related U.S. Application Data
(63) Continuation-in-part of application No. 16/167,108, filed on Oct. 22, 2018.
Application No. 16/167,108 is a continuation-in-part of application No. 16/167,131, filed on Oct. 22, 2018.

(51) Int. Cl. A61K 33/30; A61K 9/00; A61K 31/198; A61K 31/55; A61K 31/194; A61K 31/522; A61K 35/742; A61K 35/747; A61K 35/745; A61K 35/744; A61K 35/741; A61K 36/064; A61K 47/26; A61K 47/10

(52) U.S. Cl. CPC A61K 33/30; A61K 47/10; A61K 9/0043; A61K 31/198; A61K 31/55; A61K 31/194; A61K 31/522; A61K 35/742; A61K 35/747; A61K 35/745; A61K 35/744; A61K 35/741; A61K 36/064; A61K 47/26; A61K 9/0014

(57) ABSTRACT

A method of managing non-infective nasal symptoms may include formulating a topical composition for nasal administration. The formulation may include zinc, acetylcysteine, azelastine, one or more probiotics, or a combination thereof. The formulation may also include additional ingredients such as a steroid, poloxamers, or xylitol. Ingredients of the topical composition may be provided in a dry powder format within a capsule for combining with an aqueous diluent at the time of administration. The topical composition may be administered nasally.

### CROSS-REFERENCE TO RELATED APPLICATIONS

This application is a continuation-in-part of U.S. patent applications Ser. No. 16/167,108 and 16/167,131, both filed Oct. 22, 2018. U.S. patent application Ser. No. 16/167,108 and U.S. patent application Ser. No. 16/167,131 are hereby incorporated by reference herein.

### FIELD OF THE TECHNOLOGY

The present disclosure is directed to topical composition and related methods of treating non-infective nasal symptoms.

### BACKGROUND

Respiratory tract conditions are extremely common ailments of the human experience and include rhinologic conditions, infections, and other obstructions to respiration. One reason for this frequency is constant exposure of respiratory surfaces to the external environment. For example, foreign matter such as debris, microorganisms, viruses, biological matter, and even harsh environmental conditions may enter the body during respiration, irritating or infecting respiratory tract surfaces or even the internal body. Overtime, the human body has also evolved defenses designed to protect the body from this exposure. These defenses include mucous lining and immune responses such as inflammation and increased mucous production or viscosity. Diseases and abnormalities such as chronic obstructive pulmonary disease (COPD), asthma, rhinitis, and various allergies may also include undesirable triggering or modulation of such defenses, e.g., excessive immune responses that cause bronchoconstriction or excessive mucous production or thickening.

Often times an initial treatment objective of respiratory tract conditions is to relieve the obstruction and restore unobstructed respiration by increasing drainage or relieving inflammation. Further objectives may be directed to relieving discomfort or treating the underlying condition. Treatments may include localized application or action of medications, e.g., using nasal spray or metered inhaler. However, respiratory tract conditions may manifest at multiple locations complicating targeted delivery of medication where needed, thus, systemic delivery routes such as oral or intravenous administration, have also been used. Causes of respiratory tract conditions are also numerous and identification of a precise cause may be difficult, especially when multiple conditions are present.

### SUMMARY

In one aspect, a method of formulating a topical composition for nasal administration to manage non-infective nasal symptoms includes combining a dry powder including zinc or pharmaceutical salt thereof and an aqueous diluent at the time of administration to the nasal cavity. Each unit dose in the topical composition may include zinc in an amount between about 1 mg and about 30 mg.

In one example, the dry powder is provided in a capsule and combining the dry powder and the aqueous diluent comprises releasing the dry powder from the capsule and mixing the dry powder with the aqueous diluent. In an example, the aqueous diluent may be selected from water, distilled water, sterile water, water for irrigation, water for injection, or sodium chloride solution.

In one example, the dry powder is provided in a capsule and combining the dry powder and the aqueous diluent includes adding the capsule into the aqueous diluent to dissolve the capsule and then mixing the dry powder with the aqueous diluent. In an example, the aqueous diluent may be selected from water, distilled water, sterile water, water for irrigation, water for injection, or sodium chloride solution.

In any of the above or another example, the dry powder may further include sodium citrate and each unit dose of the topical composition may include sodium citrate in an amount between about 5 mg and about 150 mg.

In any of the above or another example, the dry powder further includes acetylcysteine, and wherein each unit dose of the topical composition includes acetylcysteine in an amount between about 50 mg and about 300 mg. In a further example, the dry powder further includes azelastine, and wherein each unit dose of the topical composition includes azelastine in an amount between about 100 mcg and about 1,000 mcg.

In any of the above or another example, the method further includes combining one or more of theophylline in a unit dose amount between about 20 mg and about 200 mg, budesonide in a unit dose amount between about 0.25 mg and about 4 mg, or methylprednisolone in a unit dose amount between about 1 mg and about 10 mg.

In any of the above or another example, the dry powder may further include a probiotic component comprising one or more probiotics selected from the genus Bacillus, Bifidobacteria, Enterococcus, Escherichia, Lactobacillus, Saccharomyces, or Streptococcus.

In another aspect, a method of formulating a topical composition for nasal administration to manage non-infective nasal symptoms incudes combining a dry powder including acetylcysteine and one of both of xylitol or poloxamers with an aqueous diluent. For each unit dose in the topical composition, the acetylcysteine may be combined in an amount between about 50 mg and about 300 mg and the xylitol and/or poloxamers may be combined in an amount between about 50 mg and about 400 mg.

In one example, the dry powder is provided in a capsule and combined with the aqueous diluent by releasing the dry powder from the capsule and mixing the dry powder with the aqueous diluent or adding the capsule into the aqueous diluent to dissolve the capsule and then mixing the dry powder with the aqueous diluent. In a further example, the aqueous diluent is selected from water, distilled water, sterile water, water for irrigation, water for injection, or sodium chloride solution.

In one example, the dry powder further includes azelastine HCl and is combined in a unit dose amount of between about 100 mcg and about 1,000 mcg. In a further example, the dry powder is provided in a capsule and is combined with the aqueous diluent by releasing the dry powder from the capsule and then mixing the dry powder with the aqueous diluent or by adding the capsule into the aqueous diluent to dissolve the capsule and then mixing the dry powder with the aqueous diluent. In a further example, the aqueous diluent is selected from water, distilled water, sterile water, water for irrigation, water for injection, or sodium chloride solution.

In still another aspect, a method of formulating a topical composition for nasal administration to manage non-infective nasal symptoms includes combining a dry powder including one or both of xylitol or poloxamers and a probiotic component with an aqueous diluent. The probiotic component includes one or more probiotics. For each unit dose in the topical composition, the xylitol and/or poloxamers is combined in an amount between about 50 mg and about 400 mg and the probiotic component is combined in an amount between 1 billion CFU and 80 billion CFU.

In one example, the dry powder further includes a probiotic component comprising one or more probiotics selected from the genus Bacillus, Bifidobacteria, Enterococcus, Escherichia, Lactobacillus, Saccharomyces, or Streptococcus. In a further example, the dry powder is provided in a capsule and is combined with the aqueous diluent by releasing the dry powder from the capsule and then mixing the dry powder with the aqueous diluent or by adding the capsule into the aqueous diluent to dissolve the capsule and then mixing the dry powder with the aqueous diluent. In a further example, the aqueous diluent is selected from water, distilled water, sterile water, water for irrigation, water for injection, or sodium chloride solution.

### DESCRIPTION

The present disclosure describes topical compositions and related methods of treating, e.g., managing, non-infective nasal symptoms. The topical compositions may be formulated for nasal delivery such as irrigation or nebulization, for example. In some applications, the topical compositions may be nasally administered to patients with non-infective nasal issues or medical conditions, which are issues or conditions that are not the caused by an infection. Examples include nasal symptoms, such as anosmia, sneezing, nasal congestion, and drippy or runny nose, caused by allergies, hay fever, allergic rhinitis, nonallergic rhinitis, or nasal polyps.

In some embodiments, a topical composition disclosed herein may be used as a supplemental or replacement therapy for patients who are currently using nasally delivered steroids, nasally or orally delivered antihistamines, nasally delivered anticholinergics, nasally or orally delivered mucolytics, orally delivered montelukast, or irrigation systems to clear out the nasal cavities and remove debris.

Various embodiments of the topical composition may be used to treat non-infective nasal conditions (not caused by an infection) or symptoms of non-infective conditions such as one or more of inflammation in the nasal cavity, thick-mucus secretions in nasal cavity, allergic rhinitis (runny nose), anosmia (inability to smell), or other nasal conditions or related symptoms caused by non-infective conditions.

Embodiments of the topical composition may include various components including active agents, bases, carriers, excipients, solubilization agents, dispersion agents, emulsifiers, diluents, flavoring agents, pH adjusting agents, fillers, or the like.

The topical composition and associated methods of treatment may include a pharmaceutically effective amount of an active component, which those having skill in the art will appreciate may include salts, pharmaceutical equivalents, or derivatives thereof. For brevity, however, such salts, equivalents, and derivatives may be referred to herein with respect to the active agent or class of active agent. For example, the composition may comprise azelastine, which is intended to include an equivalent pharmaceutically effective amount of azelastine hydrochloride.

Various embodiments of the topical composition may include an active component selected from one or more of a steroid, antihistamine, anticholinergic, mucolytic, zinc, probiotic, or combinations thereof.

In some embodiments, the active component of the topical composition for the treatment of non-infective nasal symptoms in a subject comprises or consists of zinc. The zinc may be or include zinc citrate, zinc HCl, another suitable zinc salt, or combination thereof. The zinc may be provided in a dry powder format for mixing with a diluent prior to administration to a subject to formulate a treatment solution. For example, a composition for formulating the treatment solution for the treatment of non-infective nasal symptoms in a subject may include a dry powder including zinc for mixing with a diluent prior to administration to the upper respiratory tract.

A unit dose of the topical composition may comprise zinc in an amount between about 1 mg and about 30 mg, such as between about 1 mg to about 120 mg, about 1 mg to about 15 mg, about 1 mg to about 6 mg, about 5 mg to about 30 mg, about 5 mg to about 20 mg, about 5 mg to about 15 mg, about 10 mg to about 30 mg, about 10 mg to about 25 mg, about 10 mg to about 20 mg, about 15 mg to about 30 mg, about 15 mg to about 25 mg, about 15 mg to about 20 mg, about 20 to about 30 mg, about 20 mg to about 25 mg, about 25 mg to about 30 mg, about 5 mg, about 10 mg, about 15 mg, about 20 mg, about 25 mg, or about 30 mg. In one embodiment, a unit dose of the topical composition comprises zinc in an amount greater than about 5 mg, greater than about 10 mg, greater than about 15 mg, greater than about 20 mg, greater than about 25 mg, or greater than about 30 mg.

In one embodiment, the dry powder including zinc of the composition may be provided in a capsule. The capsule may include an amount of zinc correspond to a unit dose of the zinc component. In another example, multiple capsules may contain a unit dose of the zinc component or a capsule may contain multiple unit dose amounts of the zinc component.

A composition for treating non-infective nasal symptoms in subject may include a dry powder including zinc for mixing with an aqueous diluent prior to administration to the upper respiratory tract. Administration to the upper respiratory tract may include intranasal administration via nasal spray, nasal drops, intranasal nebulization, or intranasal irrigation.

In some embodiments, the active component of the topical composition for the treatment of non-infective nasal symptoms in a subject comprises or consists of a probiotic. The probiotic may be provided in a dry powder format for mixing with a diluent prior to administration to a subject to formulate a treatment solution. For example, a composition for formulating the treatment solution for the treatment of non-infective nasal symptoms in a subject may include a dry powder including a probiotic component for mixing with a diluent prior to administration to the upper respiratory tract.

The probiotic component may comprise bacterial probiotic or a yeast probiotic. The probiotic component may address inflammation and/or modify immune function of the subject to reduce allergy related nasal symptoms. In various embodiments, the probiotic component comprises or consists of one or more probiotics selected from the genus Bacillus, Bifidobacteria, Enterococcus, Escherichia, Lactobacillus, Saccharomyces, or Streptococcus. For example, the topical composition may include a probiotic selected from Lactobacillus acetotolerans, Lactobacillus acidophilus, Lactobacillus alvi, Lactobacillus amylolyticus, Lactobacillus amylovorus, Lactobacillus apis, Lactobacillus backi, Lactobacillus bombicola, Lactobacillus colini, Lactobacillus crispatus, Lactobacillus delbrueckii, Lactobacillus equicursoris, Lactobacillus fornicalis, Lactobacillus gallinarum, Lactobacillus gasseri, Lactobacillus gigeriorum, Lactobacillus ginsenosidimutans, Lactobacillus hamster, Lactobacillus helsingborgensis, Lactobacillus helveticus, Lactobacillus hominis, Lactobacillus iners, Lactobacillus intestinalis, Lactobacillus jensenii, Lactobacillus jinshani, Lactobacillus johnsonii, Lactobacillus kalixensis, Lactobacillus kefiranofaciens, Lactobacillus kimbladii, Lactobacillus kitasatonis, Lactobacillus kullabergensis, Lactobacillus melliventris, Lactobacillus mulieris, Lactobacillus nasalidis, Lactobacillus panisapium, Lactobacillus paragasseri, Lactobacillus pasteurii, Lactobacillus porci, Lactobacillus psittaci, Lactobacillus raoultii, Lactobacillus rodentium, Lactobacillus rogosae, Lactobacillus sakei, Lactobacillus taiwanensis, Lactobacillus thermophilus, Lactobacillus timonensis, Lactobacillus ultunensis, Lactobacillus xujianguonis, Bifidobacterium actinocoloniiforme, Bifidobacterium adolescentis, Bifidobacterium angulatum, Bifidobacterium animalis, Bifidobacterium aquikefiri, Bifidobacterium asteroids, Bifidobacterium biavatii, Bifidobacterium bifidum, Bifidobacterium bohemicum, Bifidobacterium bombi, Bifidobacterium bourn, Bifidobacterium breve, Bifidobacterium callitrichos, Bifidobacterium catenulatum, Bifidobacterium choerinum, Bifidobacterium commune, Bifidobacterium coryneforme, Bifidobacterium cuniculi, Bifidobacterium crudilactis, Bifidobacterium denticolens, Bifidobacterium dentium, Bifidobacterium eulemuris, Bifidobacterium faecale, Bifidobacterium gallicum, Bifidobacterium gallinarum, Bifidobacterium hapali, Bifidobacterium indicum, Bifidobacterium inopinatum, Bifidobacterium kashiwanohense, Bifidobacterium lactis, Bifidobacterium lemurum, Bifidobacterium longum, Bifidobacterium magnum, Bifidobacterium merycicum, Bifidobacterium minimum, Bifidobacterium mongoliense, Bifidobacterium moukalabense, Bifidobacterium myosotis, Bifidobacterium pseudocatenulatum, Bifidobacterium pseudolongum, Bifidobacterium psychraerophilum, Bifidobacterium pullorum, Bifidobacterium reuteri, Bifidobacterium ruminantium, Bifidobacterium saguini, Bifidobacterium scardovii Bifidobacterium stellenboschense, Bifidobacterium stercoris, Bifidobacterium saeculare, Bifidobacterium subtile, Bifidobacterium thermacidophilum, Bifidobacterium thermophilum Bifidobacterium tissieri, Bifidobacterium tsurumiense, Bacillus clausii, Escherichia coli Nissle, Streptococcus thermophilus, or combination thereof. In one example, the probiotic comprises or consists of Bifidobacterium lactis, Dolosigranulum pigrum, Lacticaseibacillus casei, Lactococcus lactis, Lactobacillus sakei, Streptococcus thermophilus, or combination thereof. When included, the probiotic component may be present in a unit dose amount between about 1 billion and 80 billion CFU, such as about 5 billion CFU to 80 billion CFU, about 10 billion CFU to about 80 billion CFU, about 20 billion CFU to about 80 billion CFU, about 30 billion CFU to about 80 billion CFU, about 40 billion CFU to about 80 billion CFU, about 50 billion CFU to about 80 billion CFU, about 60 billion CFU to about 80 billion CFU, about 1 billion CFU to 50 billion CFU, about 1 billion CFU to about 20 billion CFU, about 1 billion CFU to about 10 billion CFU, about 10 billion CFU to about 50 billion CFU, about 10 billion CFU to about 30 billion CFU, about 20 billion CFU to about 50 billion CFU, or about 30 billion CFU to about 60 billion CFU. When multiple probiotics are included, the amount of probiotic may be divided among the unit dose amount.

In one embodiment, the dry powder including the probiotic of the composition may be provided in a capsule. The capsule may include an amount of probiotic correspond to a unit dose of the probiotic component. In another example, multiple capsules may contain a unit dose of the probiotic component or a capsule may contain multiple unit dose amounts of the probiotic component.

A composition for treating non-infective nasal symptoms in subject may include a dry powder including one or more probiotics for mixing with an aqueous diluent prior to administration to the upper respiratory tract. Administration to the upper respiratory tract may include intranasal administration via nasal spray, nasal drops, intranasal nebulization, or intranasal irrigation.

In some embodiments, the active component of the topical composition for the treatment of non-infective nasal symptoms in a subject comprises or consists of one or more steroids. One or more steroids of an active component may include a corticosteroid, glucocorticoid steroid, or both, for example. Corticosteroids mimic the effects of hormones that the body produces naturally in your adrenal glands. Corticosteroids can suppress inflammation and can reduce the signs and symptoms of inflammatory conditions (e.g., arthritis and asthma). Corticosteroids can also suppress the immune system. Corticosteroids can act on a number of different cells (e.g., mast cells, neutrophils, macrophages and lymphocytes) and a number of different mediators (e.g., histamine, leukotriene, and cytokine subtypes).

In various embodiments, the topical composition may include one or more steroids selected from triamcinolone (e.g., diacetate, hexacetonide, and acetonide), betamethasone (e.g., dipropionate, benzoate, sodium phosphate, acetate, and valerate), dexamethasone (e.g., dipropionate and valerate), flunisolide, prednisone (e.g., acetate), prednisolone (e.g., acetate, sodium phosphate, and tebutate), methylprednisolone (e.g., acetate and sodium succinate), fluocinolone (e.g., acetonide), budesonide, diflorasone (e.g., diacetate), halcinonide, desoximetasone (desoxymethasone), diflucortolone (e.g., valerate), flucloronide (fluocortolone acetonide), fluocinonide, fluocortolone, fluprednidene (e.g., acetate), flurandrenolide (flurandrenolone), clobetasol (e.g., propionate), clobetasone (e.g., butyrate), alclometasone, flumethasone (e.g., pivalate), fluocortolone (e.g., hexanoate), amcinonide, beclomethasone (e.g., dipropionate), fluticasone (e.g., propionate), difluprednate, prednicarbate, flurandrenolide, mometasone, and desonide.

In various embodiments, the topical composition includes one or more of the above steroids in a unit dose amount about 0.25 mg to about 10 mg, such as about 0.5 mg to about 8 mg, about 1 mg to about 6 mg, about 2 mg to about 5 mg, about 3 mg to about 5 mg, about 4 mg to about 6 mg, about 5 mg to about 7 mg, about 6 mg to about 9 mg, about 6 mg to about 10 mg, about 0.25 mg to about 5 mg, about 0.5 mg to about 5 mg, about 0.5 mg to about 3 mg, about 0.5 to about 2 mg, about 2 mg to about 3 mg, about 3 mg to about 4 mg, about 0.25 mg, about 0.5 mg, about 1 mg, about 2 mg, about 3 mg, about 4 mg, about 5 mg, about 6 mg, or about 7 mg. In a further embodiment, the topical composition includes a probiotic component in an associated amount identified above or elsewhere herein and/or zinc in an amount described herein, such as between about 1 mg and about 30 mg or any range therebetween together with a steroid described herein, in an amount described herein. The amounts of actives, excipients, diluent, and other components disclosed herein with respect to the topical composition may refer to unit or administration dose amounts, which represents the amount of the ingredient in a dose of the topical composition that is to be administered to the subject. As noted herein, the topical composition may include diluents or other ingredients that increase the total amount of matter in an administration dosage. Thus, the weight or volume of a unit dose may vary. For example, a nasal irrigation format may include additional diluent than a nebulization format. Those having skill in the art may determine suitable weights, volumes, and corresponding amounts of diluent suitable for the route of administration.

In one embodiment, the steroid comprises or consists of fluticasone. For example, the topical composition may include fluticasone in a unit dose amount about 0.5 mg to about 6 mg, about 1 mg to about 5 mg, about 2 mg to about 4 mg, about 2 mg to about 3 mg, about 3 mg to about 4 mg, or about 3 mg.

In one embodiment, the steroid comprises or consists of budesonide. For example, the topical composition may include budesonide in a unit dose amount of about 0.25 mg to about 4 mg, about 0.25 mg to about 3 mg, about 0.25 mg to about 2 mg, about 0.5 mg to about 2 mg, about 0.5 to about 1 mg, about 1 mg to about 2 mg, about 0.5 mg, about 1 mg, or about 2 mg.

In one embodiment, the steroid comprises or consists of methylprednisolone. For example, the topical composition may include methylprednisolone in a unit dose amount of about 1 mg to about 10 mg, about 2 mg to about 9 mg, about 3 mg to about 8 mg, about 4 mg to about 7 mg, about 4 mg to about 6 mg, about 4 mg to about 5 mg, about 4 mg, about 5 mg, about 6 mg, or about 8 mg. The methylprednisolone may include a methylprednisolone solution, suspension, emulsion, or powder.

As introduced above, the topical composition may include an active component comprising one or more antihistamines. Antihistamines act to reduce or block histamine receptors (e.g., H1 receptors and H2 receptors). When included, antihistamines may comprise or consist of, but are not limited to, one or more of the following: acrivastine, azelastine, bilastine, bromodiphenhydramine, brompheniramine, buclizine, carbinoxamine, cetirizine, chlorodiphenhydramine, chlorphenamine, chlorpheniramine, chlorpromazine, cimetidine, clemastine, cyclizine, cyproheptadine, desloratadine, dexbrompheniramine, dexchlorpheniramine, dimenhydrinate, dimetindene, diphenhydramine, doxylamine, ebastine, embramine, emedastine, famotidine, fexofenadine, hydroxyzine, lafutidine, levocabastine, loratadine, meclozine, mirtazapine, nizatidine, olopatadine, orphenadrine, phenindamine, pheniramine, phenyltoloxamine, promethazine, pyrilamine, quetiapine, ranitidine, roxatidine, rupatadine, tiotidine, tripelennamine, or triprolidine.

In some embodiments, the topical composition includes an active component comprising or consisting of one or more of a probiotic component in an associated amount identified above or elsewhere herein, zinc in an amount described herein, such as between about 1 mg and about 30 mg or any range therebetween zinc, or any of the steroids and associated amounts of the steroids, identified above or elsewhere herein, together with one or more of the above antihistamines in a unit dose amount about 10 mg to about 1 g, about 10 mg to about 500 mg, about 15 mg to about 300 mg, about 25 mg to about 300 mg, about 50 mg to about 250 mg, about 75 mg to about 200 mg, about 100 mg to about 900 mg, about 200 mg to about 800 mg, about 300 mg to about 700 mg, about 400 mg to about 700 mg, or about 500 mg to about 800 mg. In one embodiment, the antihistamine comprises or consists of azelastine in a unit dose amount about 100 mg to 1000 mg, about 200 mg to about 900 mg, 300 mg to about 800 mg, 400 mg to about 700 mg, 400 mg to about 600 mg, 500 mg to about 600 mg, about 400 mg, about 500 mg, or about 500 mg. In some embodiments, the topical composition may include an antihistamine in addition to the steroid and one or more of an anticholinergic, mucolytic, theophylline, sodium citrate, anti-inflammatory, or leukotriene receptor antagonist disclosed herein.

As introduced above, the topical composition may include an active component comprising one or more anticholinergics. Anticholinergics act to block the action of the neurotransmitter acetylcholine in both the central and peripheral nervous systems. In various embodiments, the topical composition includes one or more anticholinergics comprising or consisting of atropine, belladonna alkaloids, benzatropine, benztropine mesylate, biperiden, bupropion, chlorpheniramine, clemastine, darifenacin, dextromethorphan, dicyclomine, dimenhydrinate, diphenhydramine, doxacurium, doxepin, doxylamine, fesoterodine, flavoxate, glycopyrrolate, hexamethonium, hydroxyzine, hyoscyamine, ipratropium (e.g., ipratropium bromide), mecamylamine, orphenadrine, oxitropium, oxybutynin, procyclidine, propantheline, scopolamine, solifenacin, tiotropium, tolterodine, trihexyphenidyl, tropicamide, tubocurarine, or a combination thereof.

In some embodiments, the topical composition comprises or consists of any of one or more of a probiotic component in an associated amount identified above or elsewhere herein, zinc in an amount described herein, such as between about 1 mg and about 30 mg or any range therebetween zinc, or the steroids and associated amounts of the steroids, identified above or elsewhere herein, together with one or more of the above anticholinergics in a unit dose amount about 0.01 mg to about 1 mg, about 0.01 mg to about 0.1 mg, about 0.02 mg to about 0.1 mg, about 0.03 mg to about 0.1 mg, about 0.05 mg to about 0.6 mg, about 0.08 mg to about 0.5 mg, about 0.1 to about 0.5 mg, or about 0.5 mg to about 1 mg. In one embodiment, the anticholinergic comprises ipratropium. In some embodiments, the topical composition may include an anticholinergic in addition to the steroid and one or more of an antihistamine, mucolytic, theophylline, sodium citrate, anti-inflammatory, or leukotriene receptor antagonist disclosed herein.

As introduced above, the topical composition may include an active component comprising one or more mucolytics. Mucolytics loosen and clear mucus from the airways. The topical composition may include one or more mucolytics comprising or consisting of acetylcysteine, bromheksin, carbocysteine, erdosteine, guiafenesin, and iodinated glycerol, or pharmaceutically acceptable salts thereof, or a combination thereof.

In some embodiments, the topical composition includes an active component comprising or consisting one or more of a probiotic component in an associated amount identified above or elsewhere herein, zinc in an amount described herein, such as between about 1 mg and about 30 mg or any range therebetween zinc, or any of the steroids and associated amounts of the steroids, identified above or elsewhere herein, or any of the anticholinergics and associated amounts of the anticholinergics identified above or elsewhere herein, together with one or more of the above mucolytics in a unit dose amount about 5 mg to about 500 mg, about 15 mg to about 400 mg, about 50 mg to about 300 mg, about 75 mg to about 200 mg, about 75 mg to about 150 mg, about 100 mg to about 250 mg, or about 200 mg to about 500 mg. In some embodiments, the topical composition may include a mucolytic in addition to the steroid and one or more of an antihistamine, anticholinergic, theophylline, sodium citrate, anti-inflammatory, or leukotriene receptor antagonist. In one embodiment, the mucolytic comprises acetylcysteine in a unit dose amount about 15 mg to about 250 mg, about 25 mg to about 200 mg, about 50 mg to about 150 mg, about 75 mg to about 125 mg, or about 100 mg.

In various embodiments, the topical composition includes an active component including theophylline. Theophylline acts as a phosphodiesterase inhibitor, adenosine receptor blocker, and histone deacetylase activator. Mechanism of action of Theophylline appears to stem from smooth muscle relaxation (bronchodilation) and suppression of the response of the airways to stimuli (i.e. non-bronchodilator prophylactic effects). In some embodiments, the topical composition comprises of consists of any of the steroids and associated amounts of the steroids identified above and elsewhere herein and theophylline in a unit dose amount about 15 mg to about 250 mg, about 25 mg to about 200 mg, about 50 mg to about 150 mg, about 75 mg to about 125 mg, or about 100 mg. In some embodiments, the topical composition may include theophylline in addition to the steroid and one or more of sodium citrate, an antihistamine, a mucolytic, an anticholinergic, an anti-inflammatory, or a leukotriene receptor antagonist disclosed herein.

In various embodiments, the topical composition may include a component comprising or consisting of sodium citrate. Sodium citrate may include monosodium citrate, disodium citrate, or preferably trisodium citrate or more preferably sodium citrate dihydrate. In some embodiments, the topical composition includes sodium citrate and any of the steroids and associated amounts of the steroids identified above and elsewhere herein and/or theophylline, wherein the sodium citrate is present in a unit dose amount about 5 mg to about 150 mg, about 10 mg to about 100 mg, about 10 mg to about 75 mg, about 25 mg to about 50 mg, about 50 mg to about 75 mg, about 50 mg to about 100 mg, or about 75 mg to about 100 mg. In some embodiments, the topical composition may include sodium citrate and theophylline in addition to a steroid and one or more of an antihistamine, mucolytic, anticholinergic, anti-inflammatory, or leukotriene receptor antagonist disclosed herein.

As introduced above, in some embodiments, the topical composition may include an active component comprising one or more anti-inflammatories. The anti-inflammatory may comprise or consist of hydrocortisone, hydrocortisone acetate, dexamethasone 21-phosphate, fluocinolone, medrysone, methylprednisolone, prednisolone 21-phosphate, prednisolone acetate, fluoromethalone, betamethasone, triamcinolone, triamcinolone acetonide, and non-steroidal anti-inflammatories (NSAIDs) such as salicylate, indomethacin, ibuprofen, diclofenac, flurbiprofen, piroxicam indomethacin, ibuprofen, naxopren, piroxicam and nabumetone. In some embodiments, the topical composition may include any of the steroids and associated amounts of the steroids identified above and elsewhere herein and about 10 mg to about 200 mg anti-inflammatory. In some embodiments, the topical composition may include an anti-inflammatory in addition to one or more of a probiotic, zinc, steroid, antihistamine, mucolytic, anticholinergic, theophylline, sodium citrate, or leukotriene receptor antagonist disclosed herein.

In various embodiments, the topical composition may include an active component comprising one or more leukotriene receptor antagonists. Leukotriene receptor antagonist function as a leukotriene-related enzyme inhibitor or a leukotriene receptor antagonist to oppose the function of these inflammatory mediators. The leukotriene receptor antagonists may comprise or consist of one or more of montelukast, zafirlukast, zilueton, or a combination thereof. In some embodiments, the topical composition may include one or more of a probiotic component in an associated amount identified above or elsewhere herein, zinc in an amount described herein, such as between about 1 mg and about 30 mg or any range therebetween zinc, one or more antihistamines in an associated amount identified above or elsewhere herein, one or more mucolytics in an associated amount identified above or elsewhere herein, or any of the steroids and associated amounts identified above and elsewhere herein together with a one or more of a leukotriene receptor antagonist, an antihistamine, mucolytic, anticholinergic, theophylline, sodium citrate, or anti-inflammatory disclosed herein.

The topical composition may include an active agent component comprising quinine sulfate. Quinine sulfate may include equivalent amounts of active substance from quinine or other quinine salts such as quinine hydrochloride, quinine di-hydrochloride, quinine sulfate dehydrate, quinine bisulfate, or quinine gluconate. Quinine sulfate is an antimalarial drug indicated only for treatment of uncomplicated Plasmodium falciparum malaria and has been shown to be effective in geographical regions where resistance to chloroquine has been documented. In some embodiments, the topical composition includes one or more of a probiotic component in an associated amount identified above or elsewhere herein, zinc in an amount described herein, such as between about 1 mg and about 30 mg or any range therebetween zinc, one or more antihistamines in an associated amount identified above or elsewhere herein, one or more mucolytics in an associated amount identified above or elsewhere herein, or any of the steroids and associated amounts identified above and elsewhere herein and quinine sulfate in a unit dose amount about 50 mg to about 1000 mg, about 50 mg to about 700 mg, about 100 mg to about 700 mg, about 200 mg to about 500 mg, about 300 mg to about 400 mg, about 300 mg to about 700 mg, about 400 mg to about 700 mg, about 500 mg to about 700 mg, about 600 mg to about 700 mg, or about 325 mg or about 650 mg. Quinine sulfate may be combined with any steroid herein. For example, quinine sulfate may be combined with one or more steroids wherein the one or more steroids are present in a unit dose amount about 0.25 mg to about 10 mg, such as about 0.5 mg to about 8 mg, about 1 mg to about 6 mg, about 2 mg to about 5 mg, about 3 mg to about 5 mg, about 4 mg to about 6 mg, about 5 mg to about 7 mg, about 6 mg to about 9 mg, about 6 mg to about 10 mg, about 0.25 mg to about 5 mg, about 0.5 mg to about 5 mg, about 0.5 mg to about 3 mg, about 0.5 to about 2 mg, about 2 mg to about 3 mg, about 3 mg to about 4 mg, about 0.25 mg, about 0.5 mg, about 1 mg, about 2 mg, about 3 mg, about 4 mg, about 5 mg, about 6 mg, or about 7 mg. In one embodiment, the steroid comprises or consists of fluticasone, e.g., about 0.5 mg to about 6 mg, about 1 mg to about 5 mg, about 2 mg to about 4 mg, about 2 mg to about 3 mg, about 3 mg to about 4 mg, or about 3 mg fluticasone. In one embodiment, the steroid comprises or consists of budesonide, e.g., for example, about 0.25 mg to about 4 mg, about 0.25 mg to about 3 mg, about 0.25 mg to about 2 mg, about 0.5 mg to about 2 mg, about 0.5 to about 1 mg, about 1 mg to about 1.5 mg, about 1.5 mg to about 2 mg, about 1 mg to about 2 mg, about 0.5 mg, about 1 mg, or about 2 mg budesonide. In one embodiment, the steroid comprises or consists of methylprednisolone, e.g., about 1 mg to about 10 mg, about 2 mg to about 9 mg, about 3 mg to about 8 mg, about 4 mg to about 7 mg, about 4 mg to about 6 mg, about 4 mg to about 5 mg, about 4 mg, about 5 mg, about 6 mg, or about 8 mg methylprednisolone.

Quinine sulfate is commercially available in capsules for oral administration. Such capsules may contain 324 mg of the active ingredient quinine sulfate USP, equivalent to 269 mg free base and inactive ingredients: corn starch, magnesium stearate, and talc. Quinine sulfate or quinine sulfate capsules may be available in other capsule strengths. In some embodiments, the topical composition may include in addition to the steroid and one or more of an antihistamine, mucolytic, anticholinergic, anti-inflammatory, or leukotriene receptor antagonist, sodium citrate, or theophylline disclosed herein.

The topical composition may comprise one or more of the listed active or other components disclosed herein and one or more additional components including one or more pharmaceutically acceptable excipients. In other embodiments, however, the formulations consist of the one or more of the listed ingredients and one or more pharmaceutically acceptable excipients. Exemplary excipient components may assist in the release, dispersion, solubility, and/or the delivery of one or more of the active components or modify taste. For example, excipients may include one or more of diluents, dispersants, preservatives, solvents, co-solvents, wetting agents, buffering agents, humectants, permeation enhancer, emollient, sweetening agents, anti-foaming agents, thickening agents, or flavoring agents, for example. Diluents may include water, distilled water, sterile water, water for injection, sodium chloride, or saline solution, for example. The diluent may comprise an aqueous diluent or non-aqueous diluent.

The topical composition may comprise a topical preparation formulated for application to an external or internal body surface such skin or mucosal surfaces of the respiratory tract. The topical compositions may be formulated to act at the tissue surface or absorb for local action. In some embodiments, however, the topical preparations may include an aspect of systemic action.

The topical composition may include an excipient component including xylitol, poloxamers, or both. The amount of xylitol and/or poloxamers included in the topical composition may be between about 10 mg and about 1 g, such about 50 mg to about 500 mg, about 100 mg to about 400 mg, or about 150 mg to about 300 mg. In various embodiments, the topical composition includes a product sold under the name LOXASPERSE®, manufactured by PCCA (Houston, Tex.), that includes xylitol and poloxamers.

As introduced above, in some embodiments, the topical composition for the treatment of non-infective nasal symptoms in a subject includes an active component comprising or consisting of a zinc component. The zinc component may be provided alone or together with additional active or inactive components as a dry powder. A method of making a composition for treating non-infective nasal symptoms in subject comprising formulating a pharmaceutical composition comprising a dry powder including zinc for mixing with an aqueous diluent. A method of making a composition for treating non-infective nasal symptoms in subject may comprise formulating a topical composition including removing the dry powder comprising zinc from the capsule and mixing the dry powder with the aqueous diluent. A method of treating a subject for non-infective nasal symptoms may comprise administering the topical composition into the upper respiratory tract of the subject via nasal spray, nasal drops, intranasal nebulization, or intranasal irrigation. A method of treating a subject for non-infective nasal symptoms comprising supplying a capsule containing a unit dose amount of zinc dry powder for mixing with an aqueous diluent prior to administration. As noted above, the dry powder may be provided in a capsule. The amount of zinc in a capsule may correspond to a portion of a unit dose, a unit dose, or multiple unit doses. In various embodiments, a unit dose of the topical composition may comprise zinc in an amount between about 1 mg and about 30 mg, such as between about 1 mg to about 120 mg, about 1 mg to about 15 mg, about 1 mg to about 6 mg, about 5 mg to about 30 mg, about 5 mg to about 20 mg, about 5 mg to about 15 mg, about 10 mg to about 30 mg, about 10 mg to about 25 mg, about 10 mg to about 20 mg, about 15 mg to about 30 mg, about 15 mg to about 25 mg, about 15 mg to about 20 mg, about 20 to about 30 mg, about 20 mg to about 25 mg, about 25 mg to about 30 mg, about 5 mg, about 10 mg, about 15 mg, about 20 mg, about 25 mg, or about 30 mg. In one embodiment, a unit dose of the topical composition comprises zinc in an amount greater than about 5 mg, greater than about 10 mg, greater than about 15 mg, greater than about 20 mg, greater than about 25 mg, or greater than about 30 mg. The topical composition may include a diluent. The diluent may be mixed with the dry powder to formulate the topical composition in a format of a treatment solution for treating non-infective nasal symptoms. For example, formulating the topical composition may include mixing the dry powder contents of one or more capsules with a diluent. Thus, in one example, the topical composition may further include a diluent, e.g., as disclosed herein, such as an aqueous diluent, which may comprise or consist of water, distilled water, sterile water, water for irrigation, water for injection, saltwater, sodium chloride (e.g., 0.9%) or saline. In one formulation, the topical composition includes one or both of poloxamers or xylitol. For example, the combined amount of poloxamers and/or xylitol in the topical composition may be between about 10 mg and about 1 g, such as in a unit dose amount about 50 mg to about 500 mg, about 100 mg to about 400 mg, or about 150 mg to about 300 mg. In one example, poloxamers, xylitol, or mixture thereof may include LOXASPERSE®, which may be combined alone or in combination with one or more additional active ingredients. In one embodiment, the topical composition is configured to include or be a component of a combination therapy including one or more steroids, antihistamines, anticholinergics, mucolytics, sodium citrate, probiotics, or combination thereof, described herein, in any corresponding amounts described herein.

As introduced above, in some embodiments, the topical composition for the treatment of non-infective nasal symptoms in a subject includes an active component comprising or consisting of a probiotic component. The probiotic component may include any of the probiotics described herein and may be provided alone or together with additional active or inactive components as a dry powder. A method of making a composition for treating non-infective nasal symptoms in subject comprising formulating a pharmaceutical composition comprising a dry powder including one or more probiotics for mixing with an aqueous diluent. A method of making a composition for treating non-infective nasal symptoms in subject may comprise formulating a topical composition including removing the dry powder comprising the one or more probiotics from the capsule and mixing the dry powder with the aqueous diluent. A method of treating a subject for non-infective nasal symptoms may comprise administering the topical composition into the upper respiratory tract of the subject via nasal spray, nasal drops, intranasal nebulization, or intranasal irrigation. A method of treating a subject for non-infective nasal symptoms comprising supplying a capsule containing a unit dose amount of the one or more probiotics for mixing with an aqueous diluent prior to administration. As noted above, the dry powder may be provided in a capsule. The amount of probiotic in a capsule may correspond to a portion of a unit dose, a unit dose, or multiple unit doses. In various embodiments, a unit dose of the topical composition may comprise one or more probiotics in a combined unit dose amount between about 1 billion and 80 billion CFU, such as about 5 billion CFU to 80 billion CFU, about 10 billion CFU to about 80 billion CFU, about 20 billion CFU to about 80 billion CFU, about 30 billion CFU to about 80 billion CFU, about 40 billion CFU to about 80 billion CFU, about 50 billion CFU to about 80 billion CFU, about 60 billion CFU to about 80 billion CFU, about 1 billion CFU to 50 billion CFU, about 1 billion CFU to about 20 billion CFU, about 1 billion CFU to about 10 billion CFU, about 10 billion CFU to about 50 billion CFU, about 10 billion CFU to about 30 billion CFU, about 20 billion CFU to about 50 billion CFU, or about 30 billion CFU to about 60 billion CFU. The diluent may be mixed with the dry powder to formulate the topical composition in a format of a treatment solution for treating non-infective nasal symptoms. For example, formulating the topical composition may include mixing the dry powder contents of one or more capsules with a diluent. Thus, in one example, the topical composition may further include a diluent, e.g., as disclosed herein, such as an aqueous diluent, which may comprise or consist of water, distilled water, sterile water, water for irrigation, water for injection, saltwater, sodium chloride (e.g., 0.9%) or saline. In one formulation, the topical composition includes one or both of poloxamers or xylitol. For example, the combined amount of poloxamers and/or xylitol in the topical composition may be between about 10 mg and about 1 g, such as in a unit dose amount about 50 mg to about 500 mg, about 100 mg to about 400 mg, or about 150 mg to about 300 mg. In one example, poloxamers, xylitol, or mixture thereof may include LOXASPERSE®, which may be combined alone or in combination with one or more additional active ingredients. In one embodiment, the topical composition is configured to include or be a component of a combination therapy including zinc and/or one or more steroids, antihistamines, anticholinergics, mucolytics, or combination thereof, described herein, in any corresponding amounts described herein.

In some embodiments, the topical composition includes an active component comprising or consisting of a steroid selected from fluticasone, budesonide, methylprednisolone, or combination thereof. For example, the steroid may comprise about 0.5 mg to about 6 mg fluticasone, about 0.25 mg to about 4 mg budesonide, or about 1 mg to about 10 mg methylprednisolone. In a further embodiment, the topical composition may comprise or consist of one or more steroids and sodium citrate in a unit dose amount about 10 mg to about 150 mg, such about 10 to about 50 mg, about 50 mg to about 100 mg, or about 25 mg. In the above or a further embodiment, the active component further comprises about 5 mg to about 150 mg, such as between about 50 mg to about 150 mg, about 75 mg to about 125 mg, or about 100 mg, theophylline. In one example, the topical composition may further include a diluent, e.g., as disclosed herein, such as an aqueous diluent, which may comprise or consist of water, distilled water, sterile water, water for irrigation, water for injection, saltwater, sodium chloride (e.g., 0.9%) or saline. In one formulation, the topical composition includes one or both of poloxamers or xylitol. For example, the combined amount of poloxamers and/or xylitol in the topical composition may be between about 10 mg and about 1 g, such as in a unit dose amount about 50 mg to about 500 mg, about 100 mg to about 400 mg, or about 150 mg to about 300 mg. In one example, poloxamers, xylitol, or mixture thereof may include LOXASPERSE®, which may be combined alone or in combination with one or more additional active ingredients. In one embodiment, the topical composition is configured for combination therapy wherein the steroid is administered separately from the theophylline and sodium citrate.

In one embodiment, the topical composition comprises about 0.5 mg to about 6 mg fluticasone, about 50 mg to about 150 mg, such as about 50 mg to about 125 mg, theophylline, and/or about 10 mg to about 125 mg, such as about 10 mg to about 100 mg, sodium citrate. The topical composition may also include about 10 mg to about 1 g antihistamine and/or about 5 mg to 500 mg mucolytic. In one example, the antihistamine comprises about 100 mcg to about 1 mg azelastine. In this or another example, the mucolytic comprises about 15 mg to about 250 mg acetylcysteine. In one example, the topical composition may further include a diluent, e.g., as disclosed herein, such as an aqueous diluent, which may comprise or consist of water, distilled water, sterile water, water for irrigation, water for injection, saltwater, sodium chloride (e.g., 0.9%) or saline. In one formulation, the topical composition includes one or both of poloxamers or xylitol. For example, the combined amount of poloxamers and/or xylitol in the topical composition may be between about 10 mg and about 1 g, such as in a unit dose amount about 50 mg to about 500 mg, about 100 mg to about 400 mg, or about 150 mg to about 300 mg. In one example, poloxamers, xylitol, or mixture thereof may include LOXASPERSE®, which may be combined alone or in combination with one or more additional active ingredients. In one embodiment, the topical composition is configured for combination therapy wherein the steroid is administered separately from the theophylline and sodium citrate.

In one embodiment, the topical composition comprises about 0.25 mg to about 4 mg budesonide, about 20 mg to about 200 mg, such as about 50 mg to about 150 mg, or about 75 mg to about 100 mg, theophylline, and/or about 5 mg to about 150 mg, such as about 10 mg to about 50 mg, or about 50 mg to about 100 mg, sodium citrate. The topical composition may also include one or more of about 10 mg to about 1 g antihistamine or about 5 mg to 500 mg mucolytic. In one example, the antihistamine comprises about 100 mcg to about 1 mg azelastine. In this or another example, the mucolytic comprises about 15 mg to about 250 mg acetylcysteine. In one example, the topical composition may further include a diluent, e.g., as disclosed herein, such as an aqueous diluent, which may comprise or consist of water, distilled water, sterile water, water for irrigation, water for injection, saltwater, sodium chloride (e.g., 0.9%) or saline. In one formulation, the topical composition includes one or both of poloxamers or xylitol. For example, the combined amount of poloxamers and/or xylitol in the topical composition may be between about 10 mg and about 1 g, such as in a unit dose amount about 50 mg to about 500 mg, about 100 mg to about 400 mg, or about 150 mg to about 300 mg. In one example, poloxamers, xylitol, or mixture thereof may include LOXASPERSE®, which may be combined alone or in combination with one or more additional active ingredients. In one embodiment, the topical composition is configured for combination therapy wherein the steroid is administered separately from the theophylline and sodium citrate.

In one embodiment, the topical composition comprises about 1 mg to about 10 mg methylprednisolone, about 20 mg to about 200 mg, such as about 50 mg to about 150 mg, or about 75 mg to about 100 mg, theophylline, and/or about 5 mg to about 150 mg, such as about 10 mg to about 50 mg, or about 50 mg to about 100 mg, sodium citrate. In a further embodiment, the topical composition includes about 10 mg to about 1 g antihistamine or about 5 mg to 500 mg mucolytic. In one example, the antihistamine comprises about 100 mcg to about 1 mg azelastine. In this or another example, the mucolytic comprises about 15 mg to about 250 mg acetylcysteine. In one example, the topical composition may further include a diluent, e.g., as disclosed herein, such as an aqueous diluent, which may comprise or consist of water, distilled water, sterile water, water for irrigation, water for injection, saltwater, sodium chloride (e.g., 0.9%) or saline. In one formulation, the topical composition includes one or both of poloxamers or xylitol. For example, the combined amount of poloxamers and/or xylitol in the topical composition may be between about 10 mg and about 1 g, such as in a unit dose amount about 50 mg to about 500 mg, about 100 mg to about 400 mg, or about 150 mg to about 300 mg. In one example, poloxamers, xylitol, or mixture thereof may include LOXASPERSE®, which may be combined alone or in combination with one or more additional active ingredients. In one embodiment, the topical composition is configured for combination therapy wherein the steroid is administered separately from the theophylline and sodium citrate.

In one embodiment, a method of non-infective nasal symptom management includes nasal administration of a topical composition comprising a steroid. The steroid may comprise or consist of one or more steroids selected from, but not limited to: triamcinolone (e.g., diacetate, hexacetonide, and acetonide), betamethasone (e.g., dipropionate, benzoate, sodium phosphate, acetate, and valerate), dexamethasone (e.g., dipropionate and valerate), flunisolide, prednisone (e.g., acetate), prednisolone (e.g., acetate, sodium phosphate, and tebutate), methylprednisolone (e.g., acetate and sodium succinate), fluocinolone (e.g., acetonide), budesonide, diflorasone (e.g., diacetate), halcinonide, desoximetasone (desoxymethasone), diflucortolone (e.g., valerate), flucloronide (fluclorolone acetonide), fluocinonide, fluocortolone, fluprednidene (e.g., acetate), flurandrenolide (flurandrenolone), clobetasol (e.g., propionate), clobetasone (e.g., butyrate), alclometasone, flumethasone (e.g., pivalate), fluocortolone (e.g., hexanoate), amcinonide, beclometasone (e.g., dipropionate), fluticasone (e.g., propionate), difluprednate, prednicarbate, flurandrenolide, mometasone, and desonide.

In various embodiments, the topical composition or a method of non-infective nasal symptom management may include combining and/or administering the components of the topical composition for nasal administration, wherein the components include a steroid selected from one or more of the above steroids in a unit dose amount about 0.25 mg to about 10 mg, such as about 0.5 mg to about 8 mg, about 1 mg to about 6 mg, about 2 mg to about 5 mg, about 3 mg to about 5 mg, about 4 mg to about 6 mg, about 5 mg to about 7 mg, about 6 mg to about 9 mg, about 6 mg to about 10 mg, about 0.25 mg to about 5 mg, about 0.5 mg to about 5 mg, about 0.5 mg to about 3 mg, about 0.5 to about 2 mg, about 2 mg to about 3 mg, about 3 mg to about 4 mg, about 0.25 mg, about 0.5 mg, about 1 mg, about 1.25 mg, about 1.5 mg, about 1.75 mg, about 2 mg, about 3 mg, about 4 mg, about 5 mg, about 6 mg, or about 7 mg. The steroid may be combined or administered in a steroid solution, suspension, emulsion, or powder.

The topical composition may include the diluent or be combined, e.g., added together with the diluent and mixed to form a solution, mixture, emulsion, or suspension, for example, wherein the steroid and/or other actives or ingredients are mixed, dissolved, suspended, dispersed, or otherwise within the diluent. The diluent may comprise an aqueous diluent such as water, distilled water, sterile water, water for irrigation, water for injection, saltwater, sodium chloride (e.g., 0.9%) or saline.

The topical composition may be formulated for administration nasally, e.g., as a powder, by intranasal irrigation or nebulization. In various embodiments, a method of making the topical composition may include mixing the steroid with the diluent. The diluent may be mixed in an amount suitable for the manner of administration. For example, administration volumes for nebulizer solutions may typically range from about 0.2 ml to about 15 ml while irrigation volumes may typically range from about 20 ml to about 500 ml. In an above or another embodiment, the topical composition may include one or both of poloxamers or xylitol. The method may include combining the steroid, and poloxamers, xylitol, or mixture thereof with diluent and mixing. In some embodiments, the poloxamers, xylitol, or mixture thereof includes LOXASPERSE® and the method includes also combining the LOXASPERSE® with the diluent and steroid and mixing. In various embodiments, LOXASPERSE® may be added in a unit dose amount about 100 mg to 1 g, e.g., about 500 mg. Dosing may be 1 to 3 times a day or as otherwise needed.

Combining and mixing may be performed in a mixing container. In some examples, combining and mixing may beneficially be performed within an irrigation system vessel or nebulization vessel. As used herein, combining includes mixing.

In various embodiments, the steroid comprises or consists of at least one of fluticasone, budesonide, or methylprednisolone.

In one embodiment, the steroid comprises or consists of fluticasone. For example, the topical composition or the method of non-infective nasal system management may include formulating the topical composition to include fluticasone in a unit dose amount about 0.5 mg to about 6 mg, about 1 mg to about 5 mg, about 2 mg to about 4 mg, about 2 mg to about 3 mg, about 3 mg to about 4 mg, or about 3 mg. The fluconazole may include a fluconazole solution, suspension, emulsion, or powder. In one example, to formulate a nebulization or irrigation dosage formulation including 3 mg of fluticasone, the 3 mg of fluticasone powder may be combined with a suitable amount of diluent and mixed. In one embodiment, the diluent comprises distilled water. In one of the above or another embodiment, the topical composition includes one or both of poloxamers or xylitol. For example, the combined amount of poloxamers and/or xylitol may be between about 10 mg and about 1 g, such as in a unit dose amount about 50 mg to about 500 mg, about 100 mg to about 400 mg, or about 150 mg to about 300 mg. The method may include combining the steroid, and poloxamers, xylitol, or mixture thereof with diluent and mixing. In some embodiments, the poloxamers, xylitol, or mixture thereof includes LOXASPERSE® and the method includes also combining the LOXASPERSE® with the diluent and fluticasone and mixing. In various embodiments, LOXASPERSE® may be added in a unit dose amount about 100 mg to 1 g, e.g., about 500 mg. In some embodiments, the method may include combining or administering additional components of the topical composition comprising about 20 mg to about 200 mg, such as about 50 mg to about 150 mg, or about 75 mg to about 100 mg, theophylline, and/or about 5 mg to about 150 mg, such as about 10 mg to about 50 mg, or about 50 mg to about 100 mg, sodium citrate.

In one embodiment, the steroid comprises or consists of budesonide. For example, the topical composition or the method of non-infective nasal system management may include formulating the topical composition to include budesonide in a unit dose amount about 0.25 mg to about 4 mg, about 0.25 mg to about 3 mg, about 0.25 mg to about 2 mg, about 0.5 mg to about 2 mg, about 0.5 mg to about 1 mg, about 1 mg to about 2 mg, about 0.5 mg, about 1 mg, or about 2 mg. The budesonide may include a budesonide solution, suspension, emulsion, or powder. In some embodiments, the method may include formulating the topical composition and/or administering additional components of the topical composition comprising about 20 mg to about 200 mg, such as about 50 mg to about 150 mg, or about 75 mg to about 100 mg, theophylline, and/or about 5 mg to about 150 mg, such as about 10 mg to about 50 mg, or about 50 mg to about 100 mg, sodium citrate.

In various embodiments, the method may comprise utilizing the contents of one or more commercially available budesonide vials. Budesonide vials contain 2 ml of sterile liquid suspension including 0.25 mg, 0.5 mg, and 1 mg budesonide. Budesonide inhalation suspension, for example, may contain micronized budesonide, sodium chloride, disodium edetate, polysorbate 80, citric acid, tri-sodium citrate, and water for injection. In one example, the method may include combining one or more budesonide 0.5 mg-2 ml vials, 1 mg-2 ml vials, or 1 mg-2 ml vials with a suitable amount of diluent to formulate a nebulization or irrigation dosage formulation and mixing. For example, to formulate a nebulization or irrigation dosage formulation including 0.5 mg budesonide, the contents of a budesonide 0.5 mg-2 ml vial may be combined with diluent and mixed. Similarly, to formulate a nebulization or irrigation dosage formulation including 1 mg budesonide, the contents of a budesonide 1 mg-2 ml vial may be combined with diluent and mixed. Multiples of budesonide vials may also be used to make dosage formulations with higher unit doses of budesonide than provided by the contents of a vial. In some embodiments, fractions of a vial may also be used for administration/unit dose formulations with lower doses of budesonide than provide by the contents of a vial. In one embodiment, the diluent comprises distilled water. In one of the above or another embodiment, the topical composition includes one or both of poloxamers or xylitol. The method may include combining the steroid, and poloxamers, xylitol, or mixture thereof with diluent and mixing. For example, the combined amount of poloxamers and/or xylitol may be between about 10 mg and about 1 g, such as in a unit dose amount about 50 mg to about 500 mg, about 100 mg to about 400 mg, or about 150 mg to about 300 mg. In some embodiments, the poloxamers, xylitol, or mixture thereof includes LOXASPERSE® and the method includes also combining the LOXASPERSE® with the diluent and budesonide and mixing. In various embodiments, LOXASPERSE® may be added in a unit dose amount about 100 mg to 1 g, e.g., about 500 mg. In some embodiments, the method may include combining and/or administering additional components of the topical composition comprising about 20 mg to about 200 mg, such as about 50 mg to about 150 mg, or about 75 mg to about 100 mg, theophylline, and/or about 5 mg to about 150 mg, such as about 10 mg to about 50 mg, or about 50 mg to about 100 mg, sodium citrate. In one example, theophylline and sodium citrate powder may be provided in a compounded capsule. The contents of the capsule may be combined with the budesonide. In further examples, combining the components may also include combining one or more of a diluent, poloxamers, or xylitol as described herein.

In some embodiments, a method of managing non-infective nasal symptoms includes combining budesonide, a diluent, and about 15 mg to about 250 mg acetylcysteine, about 100 mcg to about 1 mg azelastine, or about 15 mg to about 250 mg theophylline. The method may further include mixing the combined budesonide, diluent, and acetylcysteine, azelastine, or theophylline to formulate a topical composition for nasal administration via inhalation powder or intranasal irrigation or nebulization. In a further embodiment, the method may include combining about 5 mg to about 150 mg, such as about 10 mg to about 30 mg, about 25 mg to about 50 mg, or about 50 mg to about 100 mg, sodium citrate. In the above or another embodiment, the method includes combining the theophylline in a unit dose amount about 20 mg to about 200 mg, such as about 50 mg to about 150 mg, or about 75 mg to about 100 mg, theophylline. The combining of the budesonide may include combining contents of one or more budesonide 0.5 mg-2 ml vials or one or more budesonide 1 mg-2 ml vials. In one example, the combining of the budesonide may include combining the contents of one or more budesonide 0.5 mg-2 ml vials and/or one or more budesonide 1 mg-2 ml vials. In the above or another example, combining the contents of a budesonide vial may include combining the contents of multiple budesonide 0.5 mg-2 ml vials, multiple budesonide 1 mg-2 ml vials, or a combination thereof. In one example, theophylline and sodium citrate powder may be provided in a compounded capsule. The contents of the capsule may be combined with the budesonide. In further examples, combining the components may also include combining one or more of a diluent, poloxamers, or xylitol as described herein. For example, the combined amount of poloxamers and/or xylitol may be between about 10 mg and about 1 g, such as in a unit dose amount about 50 mg to about 500 mg, about 100 mg to about 400 mg, or about 150 mg to about 300 mg.

In one embodiment, the steroid comprises or consists of methylprednisolone. For example, the topical composition or the method of non-infective nasal system management may include formulating the topical composition to include methylprednisolone in a unit dose amount about 1 mg to about 10 mg, about 2 mg to about 9 mg, about 3 mg to about 8 mg, about 4 mg to about 7 mg, about 4 mg to about 6 mg, about 4 mg to about 5 mg, about 4 mg, about 5 mg, about 6 mg, or about 8 mg. The methylprednisolone may include a methylprednisolone solution, suspension, emulsion, or powder. In one embodiment, the method may comprise combining methylprednisolone powder with a suitable amount of diluent to formulate a nebulization or irrigation solution and mixing. For example, about 5 mg of methylprednisolone powder may be combined with diluent and mixed. In one embodiment, the diluent comprises distilled water. In one of the above or another embodiment, the topical composition includes one or both of poloxamers or xylitol. For example, the combined amount of poloxamers and/or xylitol may be between about 10 mg and about 1 g, such as in a unit dose amount about 50 mg to about 500 mg, about 100 mg to about 400 mg, or about 150 mg to about 300 mg. The method may include combining the steroid, and poloxamers, xylitol, or mixture thereof with diluent and mixing. In some embodiments, the poloxamers, xylitol, or mixture thereof includes LOXASPERSE® and the method includes also combining the LOXASPERSE® with the diluent and methylprednisolone and mixing. In various embodiments, LOXASPERSE® may be added in a unit dose amount about 100 mg to 1 g, e.g., about 500 mg. In some embodiments, the method may include combining and/or administering additional components of the topical composition comprising about 20 mg to about 200 mg, such as about 50 mg to about 150 mg, or about 75 mg to about 100 mg, theophylline, and/or about 5 mg to about 150 mg, such as about 10 mg to about 50 mg, or about 50 mg to about 100 mg, sodium citrate. In one example, theophylline and sodium citrate powder may be provided in a compounded capsule. The contents of the capsule may be combined with the methylprednisolone. In further examples, combining the components may also include combining one or more of a diluent, poloxamers, or xylitol as described herein.

In one embodiment, the method of non-infective nasal symptom management may include formulating the topical composition to include one or more antihistamines in addition to the one or more of the above steroids in a listed amount. For example, the method may include combining with the steroid one or more antihistamines comprising acrivastine, azelastine, bilastine, bromodiphenhydramine, brompheniramine, buclizine, carbinoxamine, cetirizine, chlorodiphenhydramine, chlorphenamine, chlorpheniramine, chlorpromazine, cimetidine, clemastine, cyclizine, cyproheptadine, desloratadine, dexbrompheniramine, dexchlorpheniramine, dimenhydrinate, dimetindene, diphenhydramine, doxylamine, ebastine, embramine, emedastine, famotidine, fexofenadine, hydroxyzine, lafutidine, levocabastine, loratadine, meclozine, mirtazapine, nizatidine, olopatadine, orphenadrine, phenindamine, pheniramine, phenyltoloxamine, promethazine, pyrilamine, quetiapine, ranitidine, roxatidine, rupatadine, tiotidine, tripelennamine, or triprolidine. The antihistamines may be combined in a unit dose amount about 10 mg to about 1 g, about 10 mg to about 500 mg, about 15 mg to about 300 mg, about 25 mg to about 300 mg, about 50 mg to about 250 mg, about 75 mg to about 200 mg, about 100 mg to about 900 mg, about 200 mg to about 800 mg, about 300 mg to about 700 mg, about 400 mg to about 700 mg, or about 500 mg to about 800 mg. The antihistamine may include an antihistamine solution, suspension, emulsion, or powder. The antihistamine may be combined and mixed with the steroid and diluent as described above to formulate a topical composition comprising a inhalation powder, nebulization or irrigation dosage formulation. In some embodiments, the topical composition may include the antihistamine in addition to the steroid and one or more of a mucolytic, theophylline, sodium citrate, anticholinergic, anti-inflammatory, or leukotriene receptor antagonist disclosed herein.

In one embodiment, the antihistamine comprises or consists of azelastine powder in a unit dose amount about 100 mcg to 1000 mcg, about 200 mcg to about 900 mcg, 300 mcg to about 800 mcg, 400 mcg to about 700 mcg, 400 mcg to about 600 mcg, 500 mcg to about 600 mcg, about 200 mcg, about 400 mcg, about 500 mcg, about 600 mcg, about 800 mcg, or about 900 mcg.

In one example, a method of making the topical composition comprising a nebulization or irrigation dosage formulation comprising about 500 mcg azelastine and about 3 mg fluconazole comprises combining the contents of a capsule containing about 500 mcg of azelastine powder with the contents of a capsule containing about 3 mg fluconazole and a suitable amount of diluent, e.g., distilled water, and mixing. In a further example, the method further includes combining about 5 mg to about 150 mg, such as about 10 mg to about 30 mg, about 25 mg to about 50 mg, or about 50 mg to about 100 mg, sodium citrate. In the above or another example, the method further includes combining the theophylline in a unit dose amount about 20 mg to about 200 mg, such as about 50 mg to about 150 mg, or about 75 mg to about 100 mg, theophylline. In one example, theophylline and sodium citrate powder may be provided in a compounded capsule, which may be the same or different capsule as the azelastine and fluconazole.

In another example, a method of making the topical composition comprising a nebulization or irrigation dosage formulation comprising about 500 mcg azelastine and about 2 mg of budesonide comprises combining the contents of a capsule containing about 500 mcg of azelastine powder with the contents of two budesonide 1 mg-2 ml vials and a suitable amount of diluent, e.g., distilled water, and mixing. In still another example, a method of making the topical composition comprising a nebulization or irrigation dosage formulation comprising about 500 mcg azelastine and about 1 mg of budesonide comprises combining the contents of a capsule containing about 500 mcg of azelastine powder with the contents of a budesonide 1 mg-2 ml vial and mixing. In a further embodiment, a suitable amount of diluent, e.g., distilled water, may also be added and mixed. In yet another example, a method of making the topical composition comprising a nebulization or irrigation dosage formulation comprising about 500 mcg azelastine and about 0.5 mg of budesonide comprises combining the contents of a capsule containing about 500 mcg of azelastine powder with the contents of a budesonide 0.5 mg-2 ml vials and a suitable amount of diluent, e.g., distilled water, and mixing. In any of the above examples, the method further includes combining about 5 mg to about 150 mg, such as about 10 mg to about 30 mg, about 25 mg to about 50 mg, or about 50 mg to about 100 mg, sodium citrate. In the above or another example, the method further includes combining the theophylline in a unit dose amount about 20 mg to about 200 mg, such as about 50 mg to about 150 mg, or about 75 mg to about 100 mg, theophylline. In one example, theophylline and sodium citrate powder may be provided in a compounded capsule, which may be the same or different capsule as the azelastine.

In still yet another example, a method of making the topical composition comprising a nebulization or irrigation dosage formulation comprising about 500 mcg azelastine and about 5 mg of methylprednisolone comprises combining the contents of a capsule containing about 500 mcg of azelastine powder with the contents of a capsule containing about 5 mg of methylprednisolone and a suitable amount of diluent, e.g., distilled water, and mixing. In a further example, the method further includes combining about 5 mg to about 150 mg, such as about 10 mg to about 30 mg, about 25 mg to about 50 mg, or about 50 mg to about 100 mg, sodium citrate. In the above or another example, the method further includes combining the theophylline in a unit dose amount about 20 mg to about 200 mg, such as about 50 mg to about 150 mg, or about 75 mg to about 100 mg, theophylline. In one example, theophylline and sodium citrate powder may be provided in a compounded capsule, which may be the same or different capsule as the azelastine and/or methylprednisolone, e.g., the theophylline, sodium citrate, azelastine, and methylprednisolone may be provided in a single capsule or multiple capsules including any combination of the ingredients.

In one of the above or another embodiment, the topical composition includes steroid, antihistamine, one or both of poloxamers or xylitol. In some embodiments, the topical composition may also include a diluent. The method may include combining the steroid, antihistamine, and poloxamers, xylitol, or mixture thereof with diluent and mixing. For example, the combined amount of poloxamers and/or xylitol may be between about 10 mg and about 1 g, such as in a unit dose amount about 50 mg to about 500 mg, about 100 mg to about 400 mg, or about 150 mg to about 300 mg. In some embodiments, the poloxamers, xylitol, or mixture thereof includes LOXASPERSE® and the method includes also combining the LOXASPERSE® with the diluent, steroid, and antihistamine and mixing. In various embodiments, LOXASPERSE® may be added in a unit dose amount about 100 mg to 1 g, for example, about 500 mg. In one example, the LOXASPERSE® may be provided in a separate capsule or together with one or both of the steroid or antihistamine or another ingredient. Dosing may be 1 to 3 times a day or as otherwise needed. In a further example, the method further includes combining about 5 mg to about 150 mg, such as about 10 mg to about 30 mg, about 25 mg to about 50 mg, or about 50 mg to about 100 mg, sodium citrate. In the above or another example, the method further includes combining the theophylline in a unit dose amount about 20 mg to about 200 mg, such as about 50 mg to about 150 mg, or about 75 mg to about 100 mg, theophylline. In one example, theophylline and sodium citrate powder may be provided in a compounded capsule, which may be the same or different capsule as one or more of the additional ingredients.

In one embodiment, the method of non-infective nasal symptom management may include formulating the topical composition to include one or more mucolytics in addition to the one or more of the above steroids in a listed amount. For example, the method may include combining with the steroid one or more mucolytics comprising selected from acetylcysteine, bromheksin, carbocysteine, erdosteine, guiafenesin, and iodinated glycerol, or pharmaceutically acceptable salts thereof, or a combination thereof.

In some embodiments, the topical composition includes any of the steroids and associate amounts of steroid identified above and elsewhere herein and one or more of the above mucolytics in a unit dose amount about 5 mg to about 500 mg, about 15 mg to about 400 mg, about 50 mg to about 300 mg, about 75 mg to about 200 mg, about 75 mg to about 150 mg, about 100 mg to about 250 mg, about 200 mg to about 500 mg. The mucolytic may include a mucolytic solution, suspension, emulsion, or powder. The mucolytic may be combined and mixed with the steroid and diluent as described above to formulate a topical composition comprising an inhalation powder, a nebulization or irrigation dosage formulation. In some embodiments, the topical composition may include a mucolytic in addition to the steroid and one or more of an antihistamine, anticholinergic, theophylline, sodium citrate, anti-inflammatory, or leukotriene receptor antagonist disclosed herein.

In one embodiment, the mucolytic comprises acetylcysteine in a unit dose amount about 15 mg to about 250 mg, about 25 mg to about 200 mg, about 50 mg to about 150 mg, about 75 mg to about 125 mg, or about 100 mg.

In one example, a method of making the topical composition comprising a nebulization or irrigation dosage formulation comprising about 100 mg acetylcysteine and about 3 mg fluconazole comprises combining the contents of a capsule containing about 100 mg of acetylcysteine powder with the contents of a capsule containing about 3 mg fluconazole and a suitable amount of diluent, e.g., distilled water, and mixing. In a further example, the method further includes combining about 5 mg to about 150 mg, such as about 10 mg to about 30 mg, about 25 mg to about 50 mg, or about 50 mg to about 100 mg, sodium citrate. In the above or another example, the method further includes combining the theophylline in a unit dose amount about 20 mg to about 200 mg, such as about 50 mg to about 150 mg, or about 75 mg to about 100 mg, theophylline. In one example, theophylline and sodium citrate powder may be provided in a compounded capsule, which may be the same or different capsule as one or more of the additional ingredients.

In another example, a method of making the topical composition comprising a nebulization or irrigation dosage formulation comprising about 100 mg acetylcysteine and about 2 mg of budesonide comprises combining the contents of a capsule containing about 100 mg of acetylcysteine powder with the contents of two budesonide 1 mg-2 ml vials and a suitable amount of diluent, e.g., distilled water, and mixing. In a further example, the method further includes combining about 5 mg to about 150 mg, such as about 10 mg to about 30 mg, about 25 mg to about 50 mg, or about 50 mg to about 100 mg, sodium citrate. In the above or another example, the method further includes combining the theophylline in a unit dose amount about 20 mg to about 200 mg, such as about 50 mg to about 150 mg, or about 75 mg to about 100 mg, theophylline. In one example, theophylline and sodium citrate powder may be provided in a compounded capsule, which may be the same or different capsule as one or more of the additional ingredients.

In still another example, a method of making the topical composition comprising a nebulization or irrigation dosage formulation comprising about 100 mg acetylcysteine and about 1 mg of budesonide comprises combining the contents of a capsule containing about 100 mg of acetylcysteine powder with the contents of a budesonide 1 mg-2 ml vial, and a suitable amount of diluent, e.g., distilled water, and mixing. In a further example, the method further includes combining about 5 mg to about 150 mg, such as about 10 mg to about 30 mg, about 25 mg to about 50 mg, or about 50 mg to about 100 mg, sodium citrate. In the above or another example, the method further includes combining the theophylline in a unit dose amount about 20 mg to about 200 mg, such as about 50 mg to about 150 mg, or about 75 mg to about 100 mg, theophylline. In one example, theophylline and sodium citrate powder may be provided in a compounded capsule, which may be the same or different capsule as one or more of the additional ingredients.

In yet another example, a method of making the topical composition comprising a nebulization or irrigation dosage formulation comprising about 100 mg acetylcysteine and about 0.5 mg of budesonide comprises combining the contents of a capsule containing about 100 mg of acetylcysteine powder with the contents of a budesonide 0.5 mg-2 ml vials and a suitable amount of diluent, e.g., distilled water, and mixing. In a further example, the method further includes combining about 5 mg to about 150 mg, such as about 10 mg to about 30 mg, about 25 mg to about 50 mg, or about 50 mg to about 100 mg, sodium citrate. In the above or another example, the method further includes combining the theophylline in a unit dose amount about 20 mg to about 200 mg, such as about 50 mg to about 150 mg, or about 75 mg to about 100 mg, theophylline. In one example, theophylline and sodium citrate powder may be provided in a compounded capsule, which may be the same or different capsule as one or more of the additional ingredients.

In still yet another example, a method of making the topical composition comprising a nebulization or irrigation dosage formulation comprising about 100 mg acetylcysteine and about 5 mg of methylprednisolone comprises combining the contents of a capsule containing about 100 mg of acetylcysteine powder with the contents of a capsule containing about 5 mg of methylprednisolone and a suitable amount of diluent, e.g., distilled water, and mixing. In a further example, the method further includes combining about 5 mg to about 150 mg, such as about 10 mg to about 30 mg, about 25 mg to about 50 mg, or about 50 mg to about 100 mg, sodium citrate. In the above or another example, the method further includes combining the theophylline in a unit dose amount about 20 mg to about 200 mg, such as about 50 mg to about 150 mg, or about 75 mg to about 100 mg, theophylline. In one example, theophylline and sodium citrate powder may be provided in a compounded capsule, which may be the same or different capsule as one or more of the additional ingredients.

In one of the above or another embodiment, the topical composition includes steroid, mucolytic, diluent, one or both of sodium citrate or theophylline, and one or both of poloxamers or xylitol. For example, the combined amount of poloxamers and/or xylitol may be between about 10 mg and about 1 g, such as in a unit dose amount about 50 mg to about 500 mg, about 100 mg to about 400 mg, or about 150 mg to about 300 mg. The method may include combining the steroid, mucolytic, and poloxamers, xylitol, or mixture thereof with diluent and mixing. In one embodiment, the topical composition comprises an inhalation powder, nebulizer solution, spray solution, or irrigation solution. In some embodiments, the poloxamers, xylitol, or mixture thereof includes LOXASPERSE® and the method includes also combining the LOXASPERSE® with the diluent, steroid, and antihistamine and mixing. In various embodiments, LOXASPERSE® may be added in a unit dose amount about 100 mg to 1 g, for example, about 500 mg. In one example, the LOXASPERSE® may be provided in a separate capsule or together with one or both of the steroid or mucolytic or another ingredient such as sodium citrate and/or theophylline, when present. Dosing may be 1 to 3 times a day or as otherwise needed.

In one embodiment, the method of non-infective nasal symptom management may include formulating the topical composition to include theophylline in addition to the one or more of the above steroids in a listed amount. For example, the method may include combining theophylline with the steroid. In some embodiments, the method includes formulating the topical composition to includes any of the steroids and associate amounts of steroid identified above and elsewhere herein and theophylline in a unit dose amount about 15 mg to about 250 mg, about 25 mg to about 200 mg, about 50 mg to about 150 mg, about 75 mg to about 125 mg, or about 100 mg. Theophylline may include a theophylline solution, suspension, emulsion, or powder. The theophylline may be combined and mixed with the steroid and diluent as described above to formulate a topical composition comprising a nebulization or irrigation dosage formulation or may be administered as a inhalation powder. In some embodiments, the topical composition may include theophylline in addition to the steroid and one or more of an antihistamine, mucolytic, sodium citrate, anticholinergic, anti-inflammatory, or leukotriene receptor antagonist disclosed herein.

In one example, a method of making the topical composition comprising a nebulization or irrigation dosage formulation comprising about 100 mg theophylline and about 3 mg fluconazole comprises combining the contents of a capsule containing about 100 mg of theophylline powder with the contents of a capsule containing about 3 mg fluconazole and a suitable amount of diluent, e.g., distilled water, and mixing. In a further example, the topical composition may further include about 10 mg to about 150 mg, such about 10 to about 50 mg, about 50 mg to about 100 mg, or about 25 mg, sodium citrate. The sodium citrate may comprise a powder provided together with one or more other ingredients or separate in one or more capsules.

In another example, a method of making the topical composition comprising a nebulization or irrigation dosage formulation comprising about 100 mg theophylline and about 2 mg of budesonide comprises combining the contents of a capsule containing about 100 mg theophylline powder with the contents of two budesonide 1 mg-2 ml vials and a suitable amount of diluent, e.g., distilled water, and mixing. In a further example, the topical composition may further include about 10 mg to about 150 mg, such about 10 to about 50 mg, about 50 mg to about 100 mg, or about 25 mg, sodium citrate. The sodium citrate may comprise a powder provided together with one or more other ingredients or separate in one or more capsules.

In still another example, a method of making the topical composition comprising a nebulization or irrigation dosage formulation comprising about 100 mg theophylline and about 1 mg of budesonide comprises combining the contents of a capsule containing about 100 mg of theophylline powder with the contents of a budesonide 1 mg-2 ml vial, and a suitable amount of diluent, e.g., distilled water, and mixing. In a further example, the topical composition may further include about 10 mg to about 150 mg, such about 10 to about 50 mg, about 50 mg to about 100 mg, or about 25 mg, sodium citrate. The sodium citrate may comprise a powder provided together with one or more other ingredients or separate in one or more capsules.

In yet another example, a method of making the topical composition comprising a nebulization or irrigation dosage formulation comprising about 100 mg theophylline and about 0.5 mg of budesonide comprises combining the contents of a capsule containing about 100 mg of theophylline powder with the contents of a budesonide 0.5 mg-2 ml vials and a suitable amount of diluent, e.g., distilled water, and mixing. In a further example, the topical composition may further include about 10 mg to about 150 mg, such about 10 to about 50 mg, about 50 mg to about 100 mg, or about 25 mg, sodium citrate. The sodium citrate may comprise a powder provided together with one or more other ingredients or separate in one or more capsules.

In still yet another example, a method of making the topical composition comprising a nebulization or irrigation dosage formulation comprising about 100 mg theophylline and about 5 mg of methylprednisolone comprises combining the contents of a capsule containing about 100 mg of theophylline powder with the contents of a capsule containing about 5 mg of methylprednisolone and a suitable amount of diluent, e.g., distilled water, and mixing. In a further example, the topical composition may further include about 10 mg to about 150 mg, such about 10 to about 50 mg, about 50 mg to about 100 mg, or about 25 mg, sodium citrate. The sodium citrate may comprise a powder provided together with one or more other ingredients or separate in one or more capsules.

In one of the above or another embodiment, the topical composition includes steroid, theophylline, diluent, and one or both of poloxamers or xylitol. For example, the combined amount of poloxamers and/or xylitol may be between about 10 mg and about 1 g, such as in a unit dose amount about 50 mg to about 500 mg, about 100 mg to about 400 mg, or about 150 mg to about 300 mg. The method may include combining the steroid, theophylline, and poloxamers, xylitol, or mixture thereof with diluent and mixing. In some embodiments, the poloxamers, xylitol, or mixture thereof includes LOXASPERSE® and the method includes also combining the LOXASPERSE® with the diluent, steroid, and theophylline and mixing. In various embodiments, LOXASPERSE® may be added in a unit dose amount about 100 mg to 1 g, for example, about 500 mg. In one example, the LOXASPERSE® may be provided in a separate capsule or together with one or both of the steroid or theophylline or another ingredient. In a further example, the topical composition may further include about 10 mg to about 150 mg, such about 10 to about 50 mg, about 50 mg to about 100 mg, or about 25 mg, sodium citrate. The sodium citrate may comprise a powder provided together with one or more other ingredients or separate in one or more capsules. Dosing may be 1 to 3 times a day or as otherwise needed.

In one embodiment, the method of non-infective nasal symptom management may include formulating the topical composition to include quinine sulfate in addition to the steroid, e.g., one or more of the above steroids, in a listed amount. For example, the method may include addition of quinine sulfate with the steroid theophylline. In some embodiments, the method includes formulating the topical composition to includes any of the steroids and associated amounts of steroid identified above and elsewhere herein and quinine sulfate in a unit dose amount about 50 mg to about 1000 mg, about 50 mg to about 700 mg, about 50 mg to about 500 mg, about 50 mg to about 350 mg, about 100 mg to about 325 mg, about 100 mg to about 200 mg, about 200 mg to about 350 mg, about 250 mg to about 325 mg, about 150 mg to about 300 mg, about 275 mg to about 325 mg, about 100 mg to about 700 mg, about 200 mg to about 500 mg, about 300 mg to about 400 mg, about 300 mg to about 700 mg, about 400 mg to about 700 mg, about 500 mg to about 700 mg, about 600 mg to about 700 mg, or about 325 mg or about 650 mg. Quinine sulfate may include a quinine sulfate solution, suspension, emulsion, tablet, capsule, or powder. Quinine sulfate may include commercially available quinine sulfate, e.g., quinine sulfate solution, suspension, emulsion, capsule, table or powder. The quinine sulfate may be combined and mixed with the steroid and diluent as described above to formulate a topical composition comprising a nebulization or irrigation dosage formulation for nasal administration. In some embodiments, the topical composition may include quinine sulfate in addition to the steroid and one or more of an antihistamine, mucolytic, anticholinergic, anti-inflammatory, theophylline, or leukotriene receptor antagonist disclosed herein.

In one example, a method of making the topical composition comprising a nebulization or irrigation dosage formulation comprising up to about 325 mg quinine sulfate and about 3 mg fluconazole comprises combining the contents or an equivalent portion thereof of a 324 mg quinine sulfate capsule with the contents of a capsule containing about 3 mg fluconazole and a suitable amount of diluent, e.g., distilled water or saline solution, and mixing. Formulations for larger dosages may also be used, e.g., dosages including up to about 650 mg quinine sulfate may be formulated with two 324 mg quinine sulfate capsules. In some embodiments, the method includes combining the contents of one or more compounded capsules including quinine sulfate powder. The quinine sulfate powder may be compounded alone, with steroid, one or more other actives, and/or with xylitol and/or poloxamers. The composition may be delivered nasally as disclosed herein.

In another example, a method of making the topical composition comprising a nebulization or irrigation dosage formulation comprising up to about 325 mg quinine sulfate and about 2 mg of budesonide comprises combining the contents or equivalent portion thereof of a 324 mg quinine sulfate capsule with the contents of two budesonide 1 mg-2 ml vials and a suitable amount of diluent, e.g., distilled water, and mixing. Formulations for larger dosages may also be used, e.g., dosages including up to about 650 mg quinine sulfate may be formulated with two 324 mg quinine sulfate capsules. In some embodiments, the method includes combining the contents of one or more compounded capsules including quinine sulfate powder. The quinine sulfate powder may be compounded alone, with steroid, one or more other actives, and/or with xylitol and/or poloxamers. The composition may be delivered nasally as disclosed herein.

In still another example, a method of making the topical composition comprising a nebulization or irrigation dosage formulation comprising up to about 325 mg quinine sulfate and about 1 mg of budesonide comprises combining the contents or an equivalent portion thereof of a 324 mg quinine sulfate capsule with the contents of a budesonide 1 mg-2 ml vial, and a suitable amount of diluent, e.g., distilled water, and mixing. Formulations for larger dosages may also be used, e.g., dosages including up to about 650 mg quinine sulfate may be formulated with two 324 mg quinine sulfate capsules. In some embodiments, the method includes combining the contents of one or more compounded capsules including quinine sulfate powder. The quinine sulfate powder may be compounded alone, with steroid, one or more other actives, and/or with xylitol and/or poloxamers. In some embodiments, the method may include combining additional ingredients of the topical composition comprising about 20 mg to about 200 mg, such as about 50 mg to about 150 mg, or about 75 mg to about 100 mg, theophylline, and/or about 5 mg to about 150 mg, such as about 10 mg to about 50 mg, about 50 mg to about 100 mg, or about 25 mg, sodium citrate. In one example, theophylline and sodium citrate powder may be provided in one or more compounded capsules together or separate of the additional ingredients. The composition may be delivered nasally as disclosed herein.

In yet another example, a method of making the topical composition comprising a nebulization or irrigation dosage formulation comprising up to about 325 mg quinine sulfate and about 0.5 mg of budesonide comprises combining the contents or an equivalent portion thereof of a 324 mg quinine sulfate capsule with the contents of a budesonide 0.5 mg-2 ml vial and a suitable amount of diluent, e.g., distilled water, and mixing. Formulations for larger dosages may also be used, e.g., dosages including up to about 650 mg quinine sulfate may be formulated with two 324 mg quinine sulfate capsules. In some embodiments, the method includes combining the contents of one or more compounded capsules including quinine sulfate powder. The quinine sulfate powder may be compounded alone, with steroid, one or more other actives, and/or with xylitol and/or poloxamers. In some embodiments, the method may include combining additional ingredients of the topical composition comprising about 20 mg to about 200 mg, such as about 50 mg to about 150 mg, or about 75 mg to about 100 mg, theophylline, and/or about 5 mg to about 150 mg, such as about 10 mg to about 50 mg, about 50 mg to about 100 mg, or about 25 mg, sodium citrate. In one example, theophylline and sodium citrate powder may be provided in one or more compounded capsules together or separate of the additional ingredients. The composition may be delivered nasally as disclosed herein.

In still yet another example, a method of making the topical composition comprising a nebulization or irrigation dosage formulation comprising up to about 325 mg quinine sulfate and about 5 mg of methylprednisolone comprises combining the contents or an equivalent portion thereof of a 324 mg quinine sulfate capsule the contents of a capsule containing about 5 mg of methylprednisolone and a suitable amount of diluent, e.g., distilled water, and mixing. Formulations for larger dosages may also be used, e.g., dosages including up to about 650 mg quinine sulfate may be formulated with two 324 mg quinine sulfate capsules. In some embodiments, the method includes combining the contents of one or more compounded capsules including quinine sulfate powder. The quinine sulfate powder may be compounded alone, with steroid, one or more other actives, and/or with xylitol and/or poloxamers. In some embodiments, the method may include combining additional ingredients of the topical composition comprising about 20 mg to about 200 mg, such as about 50 mg to about 150 mg, or about 75 mg to about 100 mg, theophylline, and/or about 5 mg to about 150 mg, such as about 10 mg to about 50 mg, about 50 mg to about 100 mg, or about 25 mg, sodium citrate. In one example, theophylline and sodium citrate powder may be provided in one or more compounded capsules together or separate of the additional ingredients. The composition may be delivered nasally as disclosed herein.

In one of the above or another embodiment, the topical composition may include steroid, quinine sulfate, diluent, and one or both of poloxamers or xylitol. For example, the combined amount of poloxamers and/or xylitol may be between about 10 mg and about 1 g, such as in a unit dose amount about 50 mg to about 500 mg, about 100 mg to about 400 mg, or about 150 mg to about 300 mg. The method may include combining the steroid, quinine sulfate, and poloxamers, xylitol, or mixture thereof with diluent and mixing. In some embodiments, the poloxamers, xylitol, or mixture thereof includes LOXASPERSE® and the method includes also combining the LOXASPERSE® with the diluent, steroid, and quinine sulfate and mixing. In various embodiments, LOXASPERSE® may be added in a unit dose amount about 100 mg to 1 g, for example, about 500 mg. In some embodiments, the method includes combining the contents of one or more compounded capsules including quinine sulfate powder. The quinine sulfate powder may be compounded alone, with steroid, one or more other actives, and/or with xylitol and/or poloxamers. The composition may be delivered nasally as disclosed herein. In one example, the LOXASPERSE® may be provided in a separate capsule or together with one or both of the steroid or quinine sulfate or another ingredient. In some embodiments, the method may include combining additional ingredients of the topical composition comprising about 20 mg to about 200 mg, such as about 50 mg to about 150 mg, or about 75 mg to about 100 mg, theophylline, and/or about 5 mg to about 150 mg, such as about 10 mg to about 50 mg, about 50 mg to about 100 mg, or about 25 mg, sodium citrate. In one example, theophylline and sodium citrate powder may be provided in one or more compounded capsules together or separate of the additional ingredients. Dosing may be 1 to 3 times a day or as otherwise needed.

In one embodiment, a method of formulating a topical composition for nasal administration to manage non-infective nasal symptoms comprising combining zinc with a diluent. The zinc may be zinc citrate, zinc HCl, or other suitable zinc salt. The diluent may be an aqueous diluent. The zinc may be provided in a dry powder capsule for mixing with an aqueous diluent at the time of administration to the nasal cavity. For each unit dose in the topical composition, the topical composition may include zinc in an amount between about 1 mg and about 30 mg, such as between about 1 mg to about 120 mg, about 1 mg to about 15 mg, about 1 mg to about 6 mg, about 5 mg to about 30 mg, about 5 mg to about 20 mg, about 5 mg to about 15 mg, about 10 mg to about 30 mg, about 10 mg to about 25 mg, about 10 mg to about 20 mg, about 15 mg to about 30 mg, about 15 mg to about 25 mg, about 15 mg to about 20 mg, about 20 to about 30 mg, about 20 mg to about 25 mg, about 25 mg to about 30 mg, about 5 mg, about 10 mg, about 15 mg, about 20 mg, about 25 mg, or about 30 mg. The method may include combining one or more additional active or inactive components, such as any of those described herein, such as one or more steroids, antihistamines, anticholinergics, mucolytics, sodium citrate, probiotics, poloxamers, xylitol, or combination thereof, described herein, in any corresponding amounts described herein. In one example, the topical composition comprising zinc and an aqueous diluent. In a further example, the topical composition also includes sodium citrate. In another example, the topical composition comprises zinc, an aqueous diluent, and one or more of acetylcysteine, azelastine, theophylline, budesonide, methylprednisolone, sodium citrate, or probiotic. In a further example, the topical composition further includes xylitol and/or poloxamers. The acetylcysteine may be provided in a unit dose amount between about 50 mg and about 300 mg, such about 50 mg to about 150 mg, about 100 mg and about 300 mg, or about 200 mg and about 300 mg. In a further example, the topical composition further includes azelastine in addition to or instead of acetylcysteine. Azelastine may be provided in a unit dose amount between about 100 mcg and about 1,000 mcg, such as about 200 mcg to about 900 mcg, 300 mcg to about 800 mcg, 400 mcg to about 700 mcg, 400 mcg to about 600 mcg, 500 mcg to about 600 mcg, about 200 mcg, about 400 mcg, about 500 mcg, about 600 mcg, about 800 mcg, or about 900 mcg. In any of the above examples, the topical composition may comprise sodium citrate in a unit dose amount between about 5 mg and about 150 mg, such as about 20 mg to about 125 mg, or about 75 mg to about 150 mg, about 5 mg to about 100 mg, about 10 mg to about 50 mg, or about 50 mg to about 100. The sodium citrate may be provide instead of or in addition to another active, such as zinc, acetylcysteine, azelastine, or theophylline. For example, the topical composition may include about 20 mg to about 200 mg, such as about 50 mg to about 150 mg, or about 75 mg to about 100 mg, theophylline, and/or about 5 mg to about 150 mg, such as about 10 mg to about 50 mg, or about 50 mg to about 100 mg, sodium citrate. In any of the above examples the topical composition may include budesonide in a unit dose amount between about 0.25 mg and about 4 mg, such as about 0.25 mg to about 3 mg, about 0.25 mg to about 2 mg, about 0.5 mg to about 2 mg, about 0.5 to about 1 mg, about 1 mg to about 2 mg, about 0.5 mg, about 1 mg, or about 2 mg. In any of the above examples, the topical composition my include methylprednisolone in a unit dose amount between about 1 mg and about 10 mg, such as about 2 mg to about 9 mg, about 3 mg to about 8 mg, about 4 mg to about 7 mg, about 4 mg to about 6 mg, about 4 mg to about 5 mg, about 4 mg, about 5 mg, about 6 mg, or about 8 mg. In any of the above examples, the topical composition may comprise a probiotic as described herein. The probiotic may be present in a unit dose amount between about 1 billion and 80 billion CFU, such as about 20 billion CFU to about 80 billion CFU, about 50 billion CFU to about 80 billion CFU, about 60 billion CFU to about 80 billion CFU, about 1 billion CFU to 20 billion CFU, or about 30 billion CFU to about 50 billion CFU. The probiotic may comprise or consist of one or more probiotics selected from the genus Bacillus, Bifidobacteria, Enterococcus, Escherichia, Lactobacillus, Saccharomyces, or Streptococcus. In one example, the probiotic comprises or consists of Bifidobacterium lactis, Dolosigranulum pigrum, Lacticaseibacillus casei, Lactococcus lactis, Lactobacillus sakei, Streptococcus thermophilus, or combination thereof. In any of the above examples, the topical composition may also include one or both of poloxamers or xylitol. For example, the combined amount of poloxamers and/or xylitol may be between about 10 mg and about 1 g, such as in a unit dose amount about 50 mg to about 500 mg, about 100 mg to about 400 mg, or about 150 mg to about 300 mg. Active and inactive components may be provided in a dry powder for mixing with an aqueous diluent prior to administration to the nasal cavity, e.g. nebulizer solution, spray solution, or irrigation solution. The aqueous diluent may comprise or consist of water, distilled water, sterile water, water for irrigation, water for injection, saltwater, sodium chloride (e.g., 0.9%) or saline. In one embodiment, budesonide and/or methylprednisolone is provided in a solution or suspension including all or a part of the diluent for further mixing with additional actives or inactives. In another example, the dry powder may be administered to the nasal cavity as an inhalation powder. Dosing may be 1 to 3 times a day or as otherwise needed.

In one embodiment, a method of formulating a topical composition for nasal administration to manage non-infective nasal symptoms comprising combining acetylcysteine with a diluent. The diluent may be an aqueous diluent. The acetylcysteine may be provided in a dry powder capsule for mixing with an aqueous diluent at the time of administration to the nasal cavity. For each unit dose in the topical composition, the topical composition may include acetylcysteine in a unit dose amount between about 50 mg and about 300 mg, such about 50 mg to about 150 mg, about 100 mg and about 300 mg, or about 200 mg and about 300 mg. In a further example, the topical composition includes azelastine in addition to or instead of acetylcysteine. Azelastine may be provided in a unit dose amount between about 100 mcg and about 1,000 mcg, such as about 200 mcg to about 900 mcg, 300 mcg to about 800 mcg, 400 mcg to about 700 mcg, 400 mcg to about 600 mcg, 500 mcg to about 600 mcg, about 200 mcg, about 400 mcg, about 500 mcg, about 600 mcg, about 800 mcg, or about 900 mcg. The method may include combining one or more additional active or inactive components, such as any of those described herein, such as one or more steroids, zinc, antihistamines, anticholinergics, mucolytics, sodium citrate, probiotics, poloxamers, xylitol, or combination thereof, described herein, in any corresponding amounts described herein. In one example, the topical composition includes acetylcysteine, sodium citrate, and an aqueous diluent. The topical composition may further include azelastine. In another example, the topical composition includes acetylcysteine, azelastine, and one or both of sodium citrate or zinc. In another example, the topical composition includes acetylcysteine or both acetylcysteine and azelastine and one or both of theophylline or budesonide. In a further example, the topical composition includes one or both of zinc or sodium citrate in addition to or instead of theophylline and/or budesonide. In a further example of any of the above examples, the topical composition may include a probiotic. In one example, any of the above examples may further include xylitol and/or poloxamers. When present, zinc may be provided in a unit dose amount between about 1 mg and about 30 mg, such as between about 1 mg to about 120 mg, about 1 mg to about 15 mg, about 1 mg to about 6 mg, about 5 mg to about 30 mg, about 5 mg to about 20 mg, about 5 mg to about 15 mg, about 10 mg to about 30 mg, about 10 mg to about 25 mg, about 10 mg to about 20 mg, about 15 mg to about 30 mg, about 15 mg to about 25 mg, about 15 mg to about 20 mg, about 20 to about 30 mg, about 20 mg to about 25 mg, about 25 mg to about 30 mg, about 5 mg, about 10 mg, about 15 mg, about 20 mg, about 25 mg, or about 30 mg. When present, sodium citrate may be provided in a unit dose amount between about 5 mg and about 150 mg, such as about 20 mg to about 125 mg, or about 75 mg to about 150 mg, about 5 mg to about 100 mg, about 10 mg to about 50 mg, or about 50 mg to about 100. When present, theophylline may be provided in a unit dose amount between about 20 mg and about 200 mg, such as about 50 mg to about 150 mg, or about 75 mg to about 100 mg. When present, budesonide may be provided in a unit dose amount between about 0.25 mg and about 4 mg, such as about 0.25 mg to about 3 mg, about 0.25 mg to about 2 mg, about 0.5 mg to about 2 mg, about 0.5 to about 1 mg, about 1 mg to about 2 mg, about 0.5 mg, about 1 mg, or about 2 mg. When present, methylprednisolone may be provided in a unit dose amount between about 1 mg and about 10 mg, such as about 2 mg to about 9 mg, about 3 mg to about 8 mg, about 4 mg to about 7 mg, about 4 mg to about 6 mg, about 4 mg to about 5 mg, about 4 mg, about 5 mg, about 6 mg, or about 8 mg. When present, poloxamers and/or xylitol may be provided in a unit dose amount between about 10 mg and about 1 g, such as about 50 mg to about 500 mg, about 100 mg to about 400 mg, or about 150 mg to about 300 mg. When present, a probiotic component may be present in a unit dose amount between about 1 billion and 80 billion CFU, such as about 20 billion CFU to about 80 billion CFU, about 50 billion CFU to about 80 billion CFU, about 60 billion CFU to about 80 billion CFU, about 1 billion CFU to 20 billion CFU, or about 30 billion CFU to about 50 billion CFU. The probiotic may comprise or consist of one or more probiotics selected from the genus Bacillus, Bifidobacteria, Enterococcus, Escherichia, Lactobacillus, Saccharomyces, or Streptococcus. In one example, the probiotic comprises or consists of Bifidobacterium lactis, Dolosigranulum pigrum, Lacticaseibacillus casei, Lactococcus lactis, Lactobacillus sakei, Streptococcus thermophilus, or combination thereof. In various embodiments of the above example topical compositions, active and/or inactive components may be provided in a dry powder for mixing with an aqueous diluent prior to administration to the nasal cavity, e.g. nebulizer solution, spray solution, or irrigation solution. The aqueous diluent may comprise or consist of water, distilled water, sterile water, water for irrigation, water for injection, saltwater, sodium chloride (e.g., 0.9%) or saline. In one embodiment, budesonide and/or methylprednisolone is provided in a solution or suspension including all or a part of the diluent for further mixing with additional actives or inactives. In another example, the dry powder may be administered to the nasal cavity as an inhalation powder. Dosing may be 1 to 3 times a day or as otherwise needed.

The topical composition may comprise one or more of the listed active components disclosed herein and may further include an excipient component comprising one or more pharmaceutically acceptable excipients. In other embodiments, however, the formulations consist of the one or more of the listed ingredients and one or more pharmaceutically acceptable excipients. Exemplary excipients may assist in the release, dispersion, solubility, or the delivery of one or more of the active components or modify taste. For example, excipients may include one or more of diluents, dispersants, preservatives, solvents, co-solvents, wetting agents, buffering agents, humectants, permeation enhancer, emollient, sweetening agents, anti-foaming agents, thickening agents, or flavoring agents, for example. Diluents may include water, distilled water, sterile water, water for injection, sodium chloride, or saline solution, for example. The diluent may comprise an aqueous diluent.

In various embodiments, the method of non-infective nasal symptom management comprises nasal administering of the topical composition. For example, the topical composition may be administered via a spray in a liquid solution or dry powder, e.g., inhalation powder. In some embodiments, the topical compositions disclosed herein may be formulated without a liquid diluent for nasal administration in a powder format. In some embodiments, nasal administration may also include nasal/intranasal irrigation or nebulization dosage. Accordingly, the topical composition may comprise or be formulated as spray, powder, irrigation, or nebulizer dosage formulation configured for nasal administration. Such formulations may be configured, for example, for delivery to target sites for treatment by spray, irrigation, or nebulization. For example, the topical composition, when prepared for administration, may be formulated in a unit dose form comprising a treatment solution suitable for administration to the nasal cavity, upper respiratory tract, and in some instances lower respiratory tract. In one embodiment, the topical composition is formulated to be delivered by irrigation at the nasal cavity. In another embodiment, the topical composition is formulated to be delivered by a nebulizer to produce aerosol particles or droplets suitable for inhalation and targeted deposition of such aerosol along the respiratory tract. In some embodiments, the topical composition may be nebulized using a nebulizer configured to produce small or large aerosol particles, with respect to the particle size dispersion generated by the nebulization, e.g., using a Nasoneb, Sinustar, or other suitable nebulizer. Various embodiments may further comprise a fluid, carrier, diluent, which may include delivery vehicles, excipients, or additional active components.

In one embodiment, the topical composition is formulated into a nebulizer formulation for delivery via a small particle nebulizer device or delivery system. The small particle nebulization delivery system may be configured to nebulize the formulation, e.g., solution, to produce small particles or droplets, e.g., having aerosol characteristics, wherein the particle size of the majority of the particles or droplets formed by the nebulization is less than about 10 microns, about 8 microns, about 5 microns, or about 3 microns. For example, in some embodiments, about 60%, 70%, 80%, 90% or greater of the particles or droplets formed by the nebulization are less than about 5 microns. In these or other embodiments, the particles may be produced within a particle size dispersion wherein at least 50%, 60%, 70%, 80%, 90%, or 95% of the particles may be with about 3 microns and about 10 microns, about 3 microns and about 8 microns, about 3 microns and about 5 microns, about 5 microns and about 8 microns, about 5 microns and about 10 microns, or about 8 microns and about 10 microns.

Accordingly, a method of administering the topical composition comprising a nebulizer formulation may comprise using a small particle nebulizer delivery system and nebulizing the formulation to form small particles or droplets. The small particles may then be inhaled into the upper airway and deposit at the paranasal sinus and nasal mucosa. Compared to large particle nebulizer delivery systems, small particle nebulizer delivery systems may be used to deliver a greater fraction of active components to the pulmonary system. This may increase systemic bioavailability of the active components. However, when increased systemic bioavailability is not desirable, e.g., when such bioavailability is linked to unwanted side effects, the formulation may be prepared for and delivered by a large particle nebulizer delivery system. While any suitable small particle nebulizer delivery system or device may be used, one suitable device is a PART or Sinustar intranasal nebulizer.

In one embodiment, the topical composition comprises a nebulizer formulation for delivery via a large particle nebulizer or delivery system. The large particle nebulizer delivery system may include a nebulizer configured to generate particles or droplets wherein the majority of the particles or droplets are larger than about 5 microns, about 10 microns, about 15 microns, about 20 microns or more, such as about 23 microns. In various embodiments, nebulization with a large particle nebulizer produces aerosol particles wherein the majority of particles are greater than about 10 microns, about 15 microns, about 20 microns, or about 25 microns. In these or other embodiments, the particles may be produced within a particle size dispersion wherein at least 50%, 60%, 70%, 80%, 90%, or 95% of the particles may be within about 10 microns and about 25 microns, about 10 microns and about 20 microns, about 10 microns and about 15 microns, about 15 microns and about 25 microns, about 15 microns and about 20 microns, or about 20 microns and about 25 microns. Accordingly, a method of administering the topical composition comprising a nebulizer formulation for large particle nebulization may comprises nebulizing the nebulizer solution to form large particles. The large particles may then be inhaled into the nasal and paranasal sinus cavities and for deposition on the frontal recess/sinus, spheno-ethmoid recess, ethmoid cavity, sphenoid and maxillary sinuses, turbinates, middle meatus, and olfactory cleft. The large particle nebulizer delivery system may be configured to provide low volume, high concentration delivery of the formulation. An exemplary nebulizer device is a NasoNeb® Nasal Nebulizer. Such large particle delivery systems may be employed to deliver a deep, penetrating aerosol to the nasal and paranasal sinus cavities of the patient. Such delivery may include little to no incidental pulmonary delivery of drugs, which may otherwise occur in small particle systems, as described above. For example, in some embodiments, large particle nebulization may provide superior outcomes compared to small particle nebulization to treat the upper respiratory tract, which typically include pulmonary delivery and decreased nasal and paranasal sinus cavity disposition.

In one embodiment, the large particle nebulizer system may be used to nebulize the nebulizer solution to generate large particles for delivery to the respiratory tract via a positive pressure airstream that ensures the components of the composition reach all of the desired nasal and paranasal sinus cavities. The large particle nebulizer system may preferably deliver the large particles such that they are readily filtered by the nose to ensure a large percentage of medication is delivered upon target surfaces where intended and that little or no unintended components of the formulation are delivered to the lungs, thus, reducing the risk of unwanted complications.

In one embodiment, the large particle nebulizer system is configured to deliver a low volume treatment solution comprising the composition to ensure that the active components of the formulation stay in the nasal cavity. Accordingly, such a system may reduce waste generated by irrigation systems. In one embodiment, the large particle nebulizer system is configured to deliver 0.2-15 mL of nebulizer solution comprising the unit dose of active components for retention in the nasal and paranasal sinus cavities. In one embodiment, the large particle nebulization system may also reduce complications associated with repeated exposure to cold fluid irrigation such as exostoses of the paranasal sinus cavities by warming the solution to near room temperature upon nebulization, which may help to avoid the iatrogenic complication of exostoses from cold fluid irrigation.

Administering the nebulizer treatment solution via a large particle nebulizer system may also avoid undesirable complications that may be linked to long-term use of small particle nebulization systems, which may include vocal irritation/alterations, chronic cough, antimicrobial resistance, eosinophilic pneumonia, and reduced lung function.

According to one embodiment, the nebulizer formulation is configured for treatment of allergic rhinitis or other rhinologic conditions.

According to various embodiments, delivery of the formulation via a small particle size delivery system provides penetration of the formulation or its active components into the lower respiratory tract.

The topical composition may also be configured for nasal administration via intranasal irrigation. In such embodiments, the typical mode of administration may be in flush form or liquid stream form. An example of suitable sinus rinse delivery mechanisms include the NeilMed® Sinus Rinse Bottle, a medical syringe of about 20 to about 60 ml in size, and other squeeze bottle irrigation devices. Typically, the formulation is administered two or three times a day. Various forms of irrigation may be used such as high volume, low volume, high pressure, low pressure, or combination thereof. For example, in some embodiments, the topical composition may be administered by low volume, low pressure irrigation. In another example, the topical composition may be administered by high volume, high pressure irrigation.

Effectiveness of the composition for treatment of respiratory tract conditions wherein the composition comprises a treatment solution administered via a Nasoneb, Sinustar, or irrigation system may provide advantages over conventional azelastine hydrochloride nasal spray compositions. For example, administration of the composition via intranasal nebulization, e.g., using a Nasoneb intranasal nebulizer, or with irrigation may provide the ability to increase positive pressure associated with such irrigation or nebulization. Also, if delivered via a large particle nebulization system including a large particle nebulizer device, the composition thereof will reach the paranasal sinus area in lieu of the frontal area where a nasal spray would reach thereby providing additional and enhanced benefits.

In one embodiment, the topical composition comprises or consists of sodium citrate in a unit dose amount about 10 mg to about 150 mg, such about 10 to about 50 mg, about 50 mg to about 100 mg, or about 25 mg, and theophylline in a unit dose amount about 5 mg to about 150 mg, such as between about 50 mg to about 150 mg, about 75 mg to about 125 mg, or about 100 mg. In a further embodiment, the topical composition comprises or consists of sodium citrate and theophylline, as described above, and a diluent. The diluent may be any suitable diluent, such as those described herein, e.g., an aqueous diluent such as water, distilled water, sterile water, water for irrigation, water for injection, saltwater, sodium chloride (e.g., 0.9%) or saline. In a further embodiment, the topical composition comprises or consists of sodium citrate, theophylline, and a diluent, as described above, and poloxamers and/or xylitol, wherein the poloxamers and/or xylitol are included in a combined amount between about 10 mg and about 1 g, such as in a unit dose amount about 50 mg to about 500 mg, about 100 mg to about 400 mg, or about 150 mg to about 300 mg. In some embodiments, the topical composition may further include or exclude one or more of quinine sulfate, an antihistamine, mucolytic, anticholinergic, anti-inflammatory, or leukotriene receptor antagonist as disclosed herein. In one embodiment, all or a portion of the topical composition is administered as an inhalation powder and any remaining portion is administered as a nebulization, irrigation, or spray solution. For example, sodium citrate and theophylline may be administered as an inhalation powder and a steroid may be administered as a nasal nebulization, irrigation, or spray solution. The topical composition may be administered to treat anti-infective nasal conditions described herein, such as anosmia.

In some embodiments, the topical composition may include a combination therapy of two or more separate formulations for administration together, e.g., within about 20 minutes, within about an hour, within about 4 hours, within about 6 hours, within about 12 hours, or within about 24 hours of each other. Administration of separate formulations of the components of the topical composition as a combination therapy may also be referred to as a treatment regimen.

In one embodiment, the topical composition comprises or consists of at least one steroid selected from fluticasone, budesonide, and methylprednisolone, sodium citrate in a unit dose amount about 10 mg to about 150 mg, such about 10 to about 50 mg, about 50 mg to about 100 mg, or about 25 mg, theophylline in a unit dose amount about 5 mg to about 150 mg, such as between about 50 mg to about 150 mg, about 75 mg to about 125 mg, or about 100 mg. In some examples, the steroid may comprise or consists of about 0.5 mg to about 6 mg fluticasone, about 0.25 mg to about 4 mg budesonide, or about 1 mg to about 10 mg methylprednisolone. In a further embodiment, the topical composition comprises the steroid, sodium citrate, theophylline, and poloxamers and/or xylitol in a combined amount between about 10 mg and about 1 g, such as in a unit dose amount about 50 mg to about 500 mg, about 100 mg to about 400 mg, or about 150 mg to about 300 mg. In any of the above, the topical composition may further include a diluent, e.g., as disclosed herein. In one aspect, a method of formulating a topical composition to treat a non-infective nasal symptom in a subject includes formulating the topical composition by combining, e.g., mixing, the ingredients. In some embodiments, the topical composition may include or exclude one or more of quinine sulfate, an antihistamine, mucolytic, anticholinergic, anti-inflammatory, or leukotriene receptor antagonist disclosed herein.

A method of treating a non-infective nasal symptom in a subject may include dispensing one or more capsules containing ingredients of the topical composition for subsequent mixing with a diluent or liquid prior to administration. For example, one or more capsules containing theophylline and sodium citrate may be dispensed. If the topical composition includes additional active agents, such as one or more of quinine sulfate, steroid, antihistamine, mucolytic, anticholinergic, anti-inflammatory, or leukotriene receptor antagonist as disclosed herein, in some embodiments, such additional active agents may be included in the capsules. The capsules may be mixed with a diluent prior to administration. For example, if the topical composition also includes a steroid as described herein, the capsules may include a steroid. The capsules may also include xylitol and/or poloxamers. In an example, the capsule may include theophylline and sodium citrate and, optionally xylitol and/or poloxamers. The capsule may be opened and its contents mixed with contents of a budesonide inhalation suspension vial including 0.25 mg, 0.5 mg, or 1 mg budesonide. In one embodiment, the topical composition is configured for combination therapy wherein one or more additional active agents, such as a steroid, are administered separately from the theophylline and sodium citrate, e.g., theophylline and sodium citrate may be combined with a diluent without a steroid and administered separately from the steroid as part of a combination therapy. Such additional active agent portions may be mixed with a diluent as necessary as described herein. If present, one or both of the theophylline and sodium citrate or other active agent portions, e.g., a steroid portion, may include one or both of poloxamers or xylitol.

A method of treating a non-infective nasal symptom in a subject may include administering the topical composition to the nasal cavity or upper respiratory tract as described herein. In one embodiment, the topical composition is administered as a combination therapy wherein the steroid is administered separately from the theophylline and sodium citrate portions. In another embodiment, the topical composition does not include a steroid and a steroid is not co-administered as part of a combination therapy. The topical composition may be formulated as a nebulization, irrigation, or spray dosage form. The topical composition may be administered to a subject, e.g., human, in need to treat a non-infective nasal condition such as one or more of inflammation in the nasal cavity, thick-mucus secretions in nasal cavity, allergic rhinitis (runny nose), anosmia (inability to smell), or other nasal conditions or related symptoms caused by non-infective conditions.

It is to be appreciated that the examples, embodiments, and other descriptions herein with respect to the topical composition and related methods may specifically exclude any component or ingredient thereof described herein. In some embodiments, the topical composition does not include other components, such as resins, oils, lipids, water, organic solvents, DMSO, alcohol, fatty acids, inorganic solvents, antibodies, proteins, amino acids, nucleic acids, biological tissues, or biological compounds.

This specification has been written with reference to various non-limiting and non-exhaustive embodiments. However, it will be recognized by persons having ordinary skill in the art that various substitutions, modifications, or combinations of any of the disclosed embodiments (or portions thereof) may be made within the scope of this specification. Thus, it is contemplated and understood that this specification supports additional embodiments not expressly set forth in this specification. Such embodiments may be obtained, for example, by combining, modifying, or reorganizing any of the disclosed steps, components, elements, features, aspects, characteristics, limitations, and the like, of the various non-limiting and non-exhaustive embodiments described in this specification.

The grammatical articles “one”, “a”, “an”, and “the”, as used in this specification, are intended to include “at least one” or “one or more”, unless otherwise indicated. Thus, the articles are used in this specification to refer to one or more than one (i.e., to “at least one”) of the grammatical objects of the article. By way of example, “a component” means one or more components, and thus, possibly, more than one component is contemplated and may be employed or used in an application of the described embodiments. Further, the use of a singular noun includes the plural, and the use of a plural noun includes the singular, unless the context of the usage requires otherwise. Additionally, the grammatical conjunctions “and” and “or” are used herein according to accepted usage. By way of example, “x and y” refers to “x” and “y”. On the other hand, “x or y” refers to “x”, “y”, or both “x” and “y”.

Any numerical range recited herein includes all values and ranges from the lower value to the upper value. For example, if a concentration range is stated as 1% to 50%, it is intended that values such as 2% to 40%, 10% to 30%, 1% to 3%, or 2%, 25%, 39% and the like, are expressly enumerated in this specification. These are only examples of what is specifically intended, and all possible combinations of numerical values and ranges between and including the lowest value and the highest value enumerated are to be considered to be expressly stated in this application. Numbers modified by the term “about” are intended to include +/−10% of the number modified.

The present disclosure may be embodied in other forms without departing from the spirit or essential attributes thereof and, accordingly, reference should be had to the following claims rather than the foregoing specification as indicating the scope of the invention. Further, the illustrations of arrangements disclosed herein are intended to provide a general understanding of the various embodiments, and they are not intended to serve as a complete description. Many other arrangements will be apparent to those of skill in the art upon reviewing the above description. Other arrangements may be utilized and derived therefrom, such that logical substitutions and changes may be made without departing from the scope of this disclosure.

What is claimed is:

1. A method of formulating a topical composition for nasal administration to manage non-infective nasal symptoms, the method comprising:
combining a dry powder including zinc or pharmaceutical salt thereof and an aqueous diluent at the time of administration to the nasal cavity, where each unit dose in the topical composition includes zinc in an amount between about 1 mg and about 30 mg.

2. The method of claim 1, wherein the dry powder is provided in a capsule, and wherein combining dry powder and the aqueous diluent comprises releasing the dry powder from the capsule and mixing the dry powder with the aqueous diluent.

3. The method of claim 2, wherein the aqueous diluent comprises water, distilled water, sterile water, water for irrigation, water for injection, or sodium chloride solution.

4. The method of claim 1, wherein the dry powder is provided in a capsule, and wherein combining the dry powder and the aqueous diluent comprises adding the capsule into the aqueous diluent to dissolve the capsule and then mixing the dry powder with the aqueous diluent.

5. The method of claim 4, wherein the aqueous diluent comprises water, distilled water, sterile water, water for irrigation, water for injection, or sodium chloride solution.

6. The method of claim 1, wherein the dry powder further includes sodium citrate, and wherein each unit dose of the topical composition includes sodium citrate in an amount between about 5 mg and about 150 mg.

7. The method of claim 1, wherein the dry powder further includes acetylcysteine, and wherein each unit dose of the topical composition includes acetylcysteine in an amount between about 50 mg and about 300 mg.

8. The method of claim 7, wherein the dry powder further includes azelastine, and wherein each unit dose of the topical composition includes azelastine in an amount between about 100 mcg and about 1,000 mcg.

9. The method of claim 1, further comprising combining one or more of theophylline in a unit dose amount between about 20 mg and about 200 mg, budesonide in a unit dose amount between about 0.25 mg and about 4 mg, or methylprednisolone in a unit dose amount between about 1 mg and about 10 mg.

10. The method of claim 1, wherein the dry powder further includes a probiotic component comprising one or more probiotics selected from the genus Bacillus, Bifidobacteria, Enterococcus, Escherichia, Lactobacillus, Saccharomyces, or Streptococcus.

11. A method of formulating a topical composition for nasal administration to manage non-infective nasal symptoms, the method comprising:
combining a dry powder including acetylcysteine and one or both of xylitol or poloxamers with an aqueous diluent, wherein, for each unit dose in the topical composition, the acetylcysteine is combined in an amount between about 50 mg and about 300 mg and the xylitol and/or poloxamers is combined in an amount between about 50 mg and about 400 mg.

12. The method of claim 11, wherein the dry powder is provided in a capsule, and wherein combining the dry powder with the aqueous diluent comprises releasing the dry powder from the capsule and mixing the dry powder with the aqueous diluent or adding the capsule into the aqueous diluent to dissolve the capsule and then mixing the dry powder with the aqueous diluent.

13. The method of claim 12, wherein the aqueous diluent comprises water, distilled water, sterile water, water for irrigation, water for injection, or sodium chloride solution.

14. The method of claim 11, wherein the dry powder further includes azelastine HCl is combined in a unit dose amount between about 100 mcg and about 1,000 mcg.

15. The method of claim 15, wherein the dry powder is provided in a capsule, and wherein combining the dry powder with the aqueous diluent comprises releasing the dry powder from the capsule and mixing the dry powder with the aqueous diluent or adding the capsule into the aqueous diluent to dissolve the capsule and then mixing the dry powder with the aqueous diluent.

16. The method of claim 16, wherein the aqueous diluent comprises water, distilled water, sterile water, water for irrigation, water for injection, or sodium chloride solution.

17. A method of formulating a topical composition for nasal administration to manage non-infective nasal symptoms, the method comprising:
combining a dry powder including one or both of xylitol or poloxamers and a probiotic component with an aqueous diluent, wherein the probiotic component comprises one or more probiotics, and wherein, for each unit dose in the topical composition, the xylitol and/or poloxamers is combined in an amount between about 50 mg and about 400 mg and the probiotic component is combined in an amount between 1 billion CFU and 80 billion CFU.

18. The method of claim 17, wherein the dry powder further includes a probiotic component comprising one or more probiotics selected from the genus Bacillus, Bifidobacteria, Enterococcus, Escherichia, Lactobacillus, Saccharomyces, or Streptococcus.

19. The method of claim 18, wherein the dry powder is provided in a capsule, and wherein combining the dry powder and the aqueous diluent comprises releasing the dry powder from the capsule and mixing the dry powder with the aqueous diluent or adding the capsule into the aqueous diluent to dissolve the capsule and then mixing the dry powder with the aqueous diluent.

20. The method of claim 19, wherein the aqueous diluent comprises water, distilled water, sterile water, water for irrigation, water for injection, or sodium chloride solution.
